(19) United States
(12) Patent Application Publication
(10) Pub. No.: US 2006/0018872 A1
(43) Pub. Date: Jan. 26, 2006

(54) Poly(lactic acid) copolymer hydrogels and related methods of drug delivery

(76) Inventors: Gregory N. Tew, Amherst, MA (US); Surita Bhatia, Amherst, MA (US)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 11/154,308

(22) Filed: Jun. 16, 2005

Related U.S. Application Data
(60) Provisional application No. 60/580,045, filed on Jun. 16, 2004.

(51) Int. Cl. A61K 31/765

(52) U.S. Cl. 424/78.37

(57) ABSTRACT

A-B-A triblock copolymers tailored to produce certain embodiments that have specific properties, such as relatively high elastic modulus. Also provided are methods of designing and synthesizing such a triblock copolymer having desired properties, such non-limiting copolymers comprising a poly(ethylene oxide) block and a block selected from at least one of poly(L-lactic acid) and poly(D,L-lactic acid).

This application claims priority benefit from provisional application No. 60/580,045 filed Jun. 16, 2004, the entirety of which is incorporated herein by reference.

### BACKGROUND OF THE INVENTION

Polymers are used in medicine in applications ranging from medical devices and drug delivery to tissue regeneration. Particularly useful are hydrogels composed of biodegradable hydrophobic blocks and biocompatible hydrophilic poly(ethylene oxide) PEO. Hydrogels are a cornerstone of drug delivery and tissue regeneration technology and will be more important as progress in biomedical science continues. Polymer composition impacts hydrogel structure and properties.

Block copolymers based on lactic acid (LA) and ethylene oxide (EO) segments have attracted considerable attention over the last decade mainly due to the biodegradable nature of the polyester and the biocompatibility of EO. Common variations on this plan include the use of the enantiomers poly(L-lactic acid) (PLLA), and poly(D-lactic acid) (PDLA) instead of the racemic mixture (PLA) as well as the co-polymer poly(L-lactic-co-glycolic acid) as blocks. Diblock poly(lactic acid) (PLA) and poly(ethylene oxide) (PEO) copolymers based on these two segments were first reported in 1987 by Cohn and co-workers who studied the morphology and in vitro degradation (Younes, H. and Cohn, D. Morphological Study of Biodegradable PEO/PLA Block Copolymers. J Biomed Mater Res 21: 1301-1316, 1987). These researchers studied the biocompatibility of the copolymer including implantation into rabbits for 7, 12, 17, and 22 days. Later, Kissel and co-workers began a large program focusing on the thermal and degradation properties of PLA-PEO-PLA materials (Li, Y. X. and Kissel, T. Synthesis and Properties of Biodegradable ABA Triblock Copolymers Consisting of Poly(L-Lactic Acid) or Poly(L-Lactic-Co-Glycolic Acid) a-Blocks Attached to Central Poly(Oxyethylene) B-Blocks. J Control Release 17: 247-257, 1993). This work was followed by several reports on the solid-state characterization of the copolymers including X-ray diffraction and differential scanning calorimetry (DSC). In 1997, Kim and co-workers reported the discovery of copolymers with liquid-gel transitions at body temperature and in vivo delivery by injection, although the polymer architecture was inverted to PEO-PLA-PEO (Jeong, B., et al. Biodegradable Block Copolymers as Injectable Drug-delivery Systems. Nature 388: 860-862, 1997). This report, along with renewed interest in hydrogels, has ushered in a much broader interest in materials composed of LA and EO.

Hydrogels of PLA and PEO copolymers were first reported in 1997 and were based on the same polymer architecture as PEO triblock copolymers with polypropylene oxide (PPO) (Pluronics®, BASF). These gels utilized BAB copolymers with PEO and PLA composing the B- and A-blocks, respectively. For example, a polymer with architecture PEO₅₀₀₀-PLLA₂₀₄₀-PEO₅₀₀₀ at 25 wt % underwent a sol-gel transition upon cooling below 45° C. As a result, the authors were able to inject a warm saline solution (45° C.) subcutaneously into mice, which upon cooling to body temperature formed a gel that was used to deliver 20,000 molecular weight (MW) dextran.

Since this initial report, several other polymer compositions have been reported and specifically, the opposite ABA architecture has been shown to form hydrogels, typically at lower concentrations than the BAB copolymer. The first thermo-reversible hydrogels based on PLA-PEO-PLA polymers were reported in 2001. In these initial studies, the hydrophobic A-blocks were actually composed of both lactic acid and glycolic acid. These materials exhibited lower sol-gel transitions between 10-23° C. and upper gel-sol transitions from 27-48 degrees Celsius depending on weight concentration and the degree of polymerization (DP) of A-blocks (Lee, D. S., et al. Novel Thermoreversible Gelation of Biodegradable PLGA-block-PEO-block-PLGA Triblock Copolymers in Aqueous Solution. Macromol Rapid Commun 22: 587-592, 2001).

In all cases, sol-gel transitions have been reported by the vial inversion method, which does not provide quantitative mechanical properties of the gel. These measurements are critical to develop well-defined structure-property-activity relationships for any biomedical application. In addition, the vial inversion method simply requires the elastic modulus of the gel to be greater than 65 Pascals (Pa) and does not address the phase composition of the material (single, completely phase separated, or microphase separated). In this method, the sol-gel transition is defined as the wt % at which the solution does not flow after inverting the vial for as little as 20 seconds in some cases. The phase diagram is then explored and typically plotted as temperature (ordinate) vs. concentration (abscissa). Lee and co-workers recently reported polymers composed of D,L lactide that formed gels with a sharper gel-sol transition compared to the previous lactic acid-glycolic acid polymers (Lee, H. T. and Lee, D. S. Thermoresponsive Phase Transitions of PLA-block-PEO-block-PLA-triblock Stereo-copolymers in Aqueous Solution. Macromol Res 10: 359-364, 2002). These polymers were composed of small PEO blocks with MW of ˜1,500 and, in all cases the PLA block MW was equal to or larger than the PEO.

Within the last two years, a few mechanical rheology experiments have been reported on PLA-PEO-PLA copolymers including PMG₁₄₀₀-PEO₁₄₅₀-PMG₁₄₀₀ (PMG-poly(D,L-3-methyl-glycolide)) which reported an elastic modulus for a 27 wt % sample less than 500 Pa (Zhong, Z. Y., et al. Synthesis and Aqueous Phase Behavior of Thermoresponsive Biodegradable Poly(D,L-3-Methylglycolide)-Block-Poly(Ethylene Glycol)-Block-Poly(D,L-3-Methylglycolide) Triblock Copolymers. Macromol Chem Phys 203: 1797-1803, 2002). The molecular structure of the PMG A-blocks is represented by Formula (I), below.

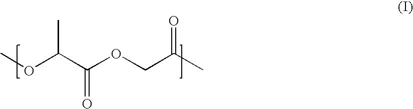

Kimura and co-workers reported hydrogel formation from stereocomplexed PLLA₁₃₀₀-PEO₄₆₀₀-PLLA₁₃₀₀ and PDLA₁₀9g-PEO₄₆₀₀-PDLA₁₀₉₀ in which 10 wt % solutions had an elastic modulus up to 1000 Pa at 37 degrees Celsius (Fujiwara, T., et al. Novel Thermo-responsive Formation of a Hydrogel by Stereo-complexation Between PLLA-PEG-PLLA and PDLA-PEG-PDLA Block Copolymers. Macromol Biosci. 1: 204-208, 2001). Solutions of either polymer independently at 10 wt % did not form a hydrogel. These reports have focused on relatively small polymers and complicated systems for initial investigations, and the data are taken at a single frequency and strain, so the dependence of modulus on frequency and strain is completely unknown.

The biological response changes every time monomer structure is changed. Hydrogels with low (<1000 Pa) and high (>1 MPa) elastic moduli are known, but those with moduli in the kPa range are not well characterized even though this modulus is of widespread biological interest. For example, although most native tissues have a nonlinear elastic response to strain, values for the modulus of several soft tissues are in this range, including human nasal cartilage (234±27 kPa) (Stockwell, R. The Chondrocyte. Adult Articular Cartilage, London, 1979 and Frank, E. H., et al. Cartilage Electromechanics-II, a Continuum Model of Cartilage Electrokinetics and Correlation with Experiments. J Biomech Eng 20: 629-639, 1987), bovine articular cartilage (990±50 kPa) (Stockwell, R. The Chondrocyte. Adult Articular Cartilage, London, 1979 and Frank, E. H., et al. Cartilage Electromechanics-II, a Continuum Model of Cartilage Electrokinetics and Correlation with Experiments. J Biomech Eng 20: 629-639, 1987), pig thoracic aorta (43.2±15 kPa) (Yu, Q. L., et al. Neutral Axis Location in Bending and Young's Modulus of Different Layers of Arterial Wall. Am J Physio 265: 52-H60, 1993), pig adventitial layer (4.72±1.7 kPa) (Yu, Q. L., et al. Neutral Axis Location in Bending and Young's Modulus of Different Layers of Arterial Wall. Am J Physio 265: H52-H60, 1993), right lobe of human liver (270±10 kPa) (Carter, F. J., et al. Measurements and Modelling of the Compliance of Human and Porcine Organs. Med Image Analysis 5: 231-236, 2001), canine kidney cortex and medulla (˜10 kPa) (Erkamp, R. Q. et al., Measuring the Elastic Modulus of Small Tissue Samples. Ultrasonic Imaging 20: 17-28, 1998), and nucleus pulposus and eye lens (˜10³ Pa) (Erkamp, R. Q. et al., Measuring the Elastic Modulus of Small Tissue Samples. Ultrasonic Imaging 20: 17-28, 1998). For scaffolding applications, it is often desirable to match the mechanical properties of the polymer matrix to those of the surrounding tissue (Hutmacher, D. W. Scaffold Design and Fabrication Technologies for Engineering Tissues: State of the Art and Future Perspectives. J Biomater Sci Polym. Ed 12: 107-124, 2001).

### SUMMARY OF THE INVENTION

In light of the foregoing, it is an object of the present invention to provide a range of triblock copolymers, of the type described herein, and/or method(s) for the preparation and subsequent use, including but not limited to the delivery and release of various pharmaceutical or therapeutic agents, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It is an object of the present invention, in contrast to the prior art, to provide ABA triblock copolymer comprising a PEO block and hydrophobic A blocks comprising but not limited to PLA having a molecular weight or degree of polymerization less than that of the PEO block.

It can also be an object of the present invention to provide such ABA triblock copolymers with elastic moduli and various other physical or mechanical properties comparable to tissues of biological or pharmaceutical interest.

It can also be an object of the present invention to establish structure-property relationships of gels formed from such copolymeric compounds.

It can also be an object of the present invention to provide a range of copolymeric compounds which can vary by degree of A block crystallinity, length and/or molecular weight, such compounds and/or variations as can be designed for a particular drug release profile.

Other objects, features, benefits and advantages of the present invention will be apparent from this summary, and the following descriptions of certain embodiments, and will be readily apparent to those skilled in the art having knowledge of various polymeric or gel systems and their use in the delivery and release of therapeutic agents incorporated therewith. Such objects, features, benefits and advantages will be apparent from the above as taken into conjunction with the accompanying examples, data, figures and all reasonable inferences to be drawn therefrom.

In part, the invention can provide an A-B-A triblock copolymer tailored to produce that have specific properties, each A block of a formula II, 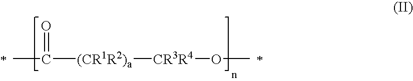
where R¹, R², R³ and R⁴ are the same or different moieties, and are selected from hydrogen, C₁-about C₆ alkyl, aryl, and X, where X═Cl, Br, F, and I; a is an integer from 0 to about 6 and n is an integer from about 10, 15 and/or 20 . . . to about 50 . . . 100 . . . and/or 300, and wherein the B block comprises poly(ethylene oxide).

In other embodiments, the present invention provides A-PEO-A triblock copolymers tailored to produce stiff hydrogels that have specific properties, such as but not limited to relatively high elastic modulus, where the A blocks comprise a polyester component selected from poly(lactic acid), poly(3-hydroxybutyrate), poly(4-hydroxybutyrate), poly(3-hydroxyvalerate), poly(caprolactone), poly(valerolactone) and mixtures thereof. In certain embodiments, at least one A block comprises poly(lactic acid).

In one embodiment, the present invention provides biocompatible polymers with controllable elastic modulus in excess of about 10 kPa. In preferred embodiments the present invention compositions and methods for making hydrogel scaffolds with modulus matched to a variety of tissues requiring regeneration including elastic cartilage, kidney and liver, as well as nucleus pulposus. In further embodiments, the hydrophobic domains can be designed to provide reservoirs for storing and then delivering therapeutic agents.

In certain embodiments, the present invention provides strong physically cross-linked hydrogels useful for widespread tissue engineering applications. Preferably a single polymer architecture is provided in which the mechanical and biological properties can be controlled over a very wide range, useful in a variety of biological applications, including those that require low or high modulus.

In certain other embodiments, the invention provides a triblock copolymer comprising about 42-about 83 wt % poly(ethylene oxide) and about 17-about 58 wt % poly(lactic acid), wherein the elastic modulus at 0.1 Hz of a hydrogel formed from an aqueous medium of about 10-about 30 wt % of the polymer is about 0.1-about 10 KPa. In a further embodiment, the invention provides a triblock copolymer comprising about 42-about 83 wt % poly(ethylene oxide) and about 17-about 58 wt % poly(lactic acid), wherein the elastic modulus at 0.1 Hz of a hydrogel formed from an aqueous medium of about 10 wt %-about 50 wt % of the polymer is about 0.1-about 1,000 KPa. In another embodiment, the invention provides a biodegradable triblock copolymer having a ratio of the degree of polymerization of poly(ethylene oxide) to the degree of polymerization of poly(lactic acid) in the range of about 1.2-about 7.8.

In other embodiments, the invention provides an A-B-A triblock copolymer having A blocks comprising about 17-about 58 wt % of a poly(ester) of formula (II), 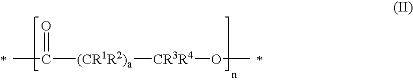
where R¹, R², R³ and R⁴ are independently selected from hydrogen, C₁-about C₆ alkyl, aryl, and X, where X═Cl, Br, F, and I, a is an integer from 0 to about 6 and n is an integer from about 10, 15 and/or 20 . . . to about 50 . . . 100 and/or 300 and having a B block comprising about 42-about 83 wt % poly(ethylene oxide), wherein the elastic modulus at 0.1 Hz of a hydrogel formed from a solution of about 10-about 50 wt % of the polymer is about 0.1-about 1,000 KPa.

In part, the present invention can also be directed to an ABA triblock copolymer compound comprising poly(lactic acid) A blocks, each A block comprising (C(O)CH(CH₃)O)ₙ wherein n is an integer ranging from about 10, 15 and/or 20 . . . to about 45 . . . and/or 50; and a B block comprising PEO, where a degree of polymerization of poly(lactic acid) ranges from about 2.0 to about 8.0. Regardless of any such numerical ratio, the degree of polymerization of poly(lactic acid) can range from about 25 and/or 30 . . . to about 50 . . . and/or 75, and independently, the molecular weight of PEO can range from about 4,000 to about 16,000. In certain embodiments, at least one A block can at least partially comprise poly(L-lactic acid). Likewise, without regard to A block composition, such a compound can comprise a hydrogel in an aqueous medium, such that the hydrogel has an elastic modulus in the kPa (e.g., about 10 to about 30 . . . 50 . . . 100 kPa) range. Such a hydrogel can further comprise a therapeutic agent with an interactive affinity (e.g., a chemical attraction and/or physical compatibility with the hydrophobic characteristic of a PLA block in an aqueous medium) for the A block of such a compound. Without limitation, such agents include but are not limited to the hydrophobic pharmaceutical compounds described herein, such compounds as can have an interactive binding or bonding affinity for an A block polymeric component of the sort described herein.

In a further embodiment, the invention provides a biodegradable polymeric drug delivery system having a ratio of the degree of polymerization of poly(ethylene oxide) to the degree of polymerization of poly(lactic acid) selected to provide a desired delivery rate based on the hydrophobicity of a drug. In another aspect, the invention provides a method of a designing a biodegradable triblock copolymer having desired properties, comprising selecting a polymer molecular weight, selecting a poly(ethylene oxide) block length, selecting a ratio of the degree of 10 polymerization of poly(ethylene oxide) to the degree of polymerization of poly(lactic acid), and selecting a relative content of poly(L-lactic acid) and poly(D,L-lactic acid).

In part, the present invention can also provide a method of using the degree of crystallinity of one or more of the present ABA triblock copolymer compounds to affect drug release. Without limitation, such a method can comprise (1) providing a PLA-PEO-PLA triblock copolymer compound comprising a PLA block comprising at least one of L-lactic acid monomers, D-lactic acid monomers and a combination thereof, such a poly(lactic acid) block having a degree of crystallinity, in an aqueous medium and an amount at least partially sufficient for gel formation; and (2) contacting such a compound with a therapeutic agent having an affinity for a poly(lactic acid) block. In certain embodiments, as discussed more fully below, an increase in poly(L-lactic acid) content can increase the rate of release of the therapeutic agent from the copolymer over time. Conversely, an increasingly racemic mixture of lactic acid monomers can decrease the rate of release.

With respect to either the compounds, therapeutic compositions and/or methods of the present invention, the moieties, block components and agents can suitably comprise, consist of, or consist essentially of any of the aforementioned substituents and monomers thereof. Each such copolymeric compound or moiety/substituent or monomer thereof is compositionally distinguishable, characteristically contrasted and can be practiced in conjunction with the present invention separate and apart from another. Accordingly, it should also be understood that the inventive compounds, compositions and/or methods, as illustratively disclosed herein, can be practiced or utilized in the absence of any one compound, monomeric moiety and/or substituent, or step which may or may not be disclosed, referenced or inferred herein, the absence of which may or may not be specifically disclosed, referenced or incurred herein.

In certain embodiments, such a method can also comprise synthesizing such a triblock copolymer, such method(s)as can comprise a variety of methods known in the art for ring-opening polymerization of cyclic lactones including but not limited to using a catalyst selected from the group consisting of Zn, CaH₂, SnO, SnO₂, SnCl₂, GeO₂, Al (OiPr)₃, Yt (alkoxides)₃, Na, potassium t-butoxide, Sn (triflate)₂, a N-heterocyclic carbene and a single site nickel catalyst.

### BRIEF DESCRIPTION OF THE DRAWINGS

FIG. 1A is a graphical presentation of the results of elastic modulus tests for hydrogels formed from samples C2-1, C2-2, and C2-3 at 20 wt %, 16 wt %, and 16 wt %, respectively, at 25 degrees Celsius.

FIG. 1B is a graphical presentation of the results of elastic modulus tests for hydrogels formed from samples C2-1, C2-2, and C2-3 at 20 wt %, 16 wt %, and 16 wt %, respectively, at 37 degrees Celsius.

FIG. 2A is a graphical representation of the temperature dependence of elastic modulus of hydrogels formed from sample C2-1.

FIG. 2B is a graphical representation of the temperature dependence of elastic modulus of hydrogels formed from sample C2-3.

FIG. 3A is a graphical representation of the viscoelastic moduli for hydrogels formed from sample C2-1, T=37° C.

FIG. 3B is a graphical representation of the viscoelastic moduli for hydrogels formed from sample C2-3, T=37° C.

FIG. 3C is a graphical representation of the viscoelastic moduli for hydrogels formed from sample C2-1, T=50° C., showing gelation (G′>G′).

FIG. 4A is a graphical representation of the elastic modulus for hydrogels formed from triblocks with PDLLA and PLLA endblocks, with the same DP of PLA: 60 units.

FIG. 4B is a graphical representation of the elastic modulus for hydrogels formed from triblocks with PDLLA and PLLA endblocks, with the same DP of PLA: 72 units.

FIG. 4C shows dependence of the storage modulus on the stereochemistry of the polymer chains. The stereoregular polymer has an storage modulus greater than 10 kPa, while the racemic version is much less stiff. Both materials are 25 wt % polymer in water.

FIGS. 4D-E show (D) stereoregular polymer with DP_{LA}=72 has a higher storage modulus than loss modulus (G′>G″) indicating gel formation; (E) A stereorandom polymer with DP_{LA}=72 does not form a gel since the storage modulus is comparable to the loss modulus (G′˜G″).

FIG. 4F shows a gel formed from a stereregular polymer with DP_{LA}=58 has a higher storage modulus than a gel formed from a stereorandom polymer with DP_{LA}=60. This is true even though the racemic polymer is two units longer than the stereoregular one.

FIGS. 4G-H show (G) The powder sample shows peaks at 2θ≈19° and 23° corresponding to the crystalline regions of both PEO and PLLA, and at 2θ≈17° corresponding to crystalline PLLA; (H) The PLLA gel sample shows only peaks corresponding to PLLA at 2θ≈17° and 19°. The PRLA sample shows no crystallinity as expected.

FIG. 5A is a Raman spectrum of a PLLA gel showing the carbonyl stretch.

FIG. 5B is a Raman spectrum of PLLA gel showing the bending region. Both the carbonyl and signals at 398 cm⁻¹ and 417 cm⁻¹ are consistent with crystallinity but not amorphous PLA.

FIG. 6A is a graphical representation of results of small angle neutron scattering (SANS) tests showing spectra at 25° C. for PLLA-PEO-PLLA solutions and gels with increasing PLLA block length at a polymer concentration of 0.5 wt %.

FIG. 6B is a graphical representation of results of small angle neutron scattering (SANS) tests showing spectra at 25° C. for PLLA-PEO-PLLA solutions and gels with increasing PLLA block length at a polymer concentration of 10.0 wt %.

FIG. 6C is a graphical representation of results of small angle neutron scattering (SANS) tests showing spectra at 25° C. for PLLA-PEO-PLLA solutions and gels with increasing PLLA block length at a polymer concentration of 22.0 wt %.

FIG. 7A is a graphical representation of the release profile methyl paraben from PLA-PEO-PLA gels, loaded in systems with PLLA endblocks (1L) and PDLLA endblocks (2D/L).

FIG. 7B is a graphical representation of the release profile indomethacin from PLA-PEO-PLA gels, loaded in systems with PLLA endblocks (1L) and PDLLA endblocks (2D/L).

FIG. 8 is an illustrative representation of gel formation. As the solution concentration of polymer is increased, micelles are initially formed. These associate through close-packing or network bridging. In the case of crystalline domains, the hydrophobic domains may get larger and change shape to allow better chain packing.

FIG. 9A is a graphical representation of absorption data collected using molecular probes to determine the critical micellar concentration.

FIG. 9B is a graphical representation of fluorescence data collected using molecular probes to determine the critical micellar concentration.

FIG. 10A is a schematic diagram of the behavior of a phase separated gel and single gel. The phase separated gel retains structure and properties much longer.

FIG. 10B is a schematic diagram illustrating surface plasmon resonance spectroscopy (SPRS) experimental set-up.

FIG. 11 is a schematic diagram of the gradient release of two drugs. One drug is hydrophilic and released quickly since it is in the hydrophilic domains while the other drug, which is hydrophobic, experiences more hindered release.

FIG. 12 is a schematic diagram of an embodiment of a method 100 of designing and synthesizing a triblock copolymer having desired properties, comprising the steps of selecting a polymer molecular weight 110; selecting a poly(ethylene oxide) block length 120; selecting a ratio of the degree of polymerization of poly(ethylene oxide) to the degree of polymerization of poly(lactic acid) 130; selecting the relative content of poly(L-lactic acid) and poly(D,L-lactic acid) 140 and synthesizing the triblock copolymer.

FIG. 13 shows SANS spectra for sample 72R at the concentrations shown.

FIG. 14 correlates SANS spectra with change in PLA block length at a fixed concentration (30wt %).

FIG. 15 shows SANS spectra for sample 72L at the concentrations shown.

FIG. 16 correlates SANS spectra with change in PLA block length at a fixed concentration (30 wt %).

FIG. 17 fits an illustrative, non-limiting model of this invention to data obtained for samples 72R, 88R and 92R.

FIG. 18 fits an illustrative, non-limiting model of this invention to data obtained for sample 58L at the concentrations shown.

FIG. 19 can be used to obtain values of parameter ε, by fitting equation (4) to scattering data, neglecting the interactions at low concentration, the scattered intensity at small values of q can be expressed as

$$I ( q , ϕ << 1 ) = I ( q \to 0 ) exp ( - \frac{{q}^{2} {R}_{g}^{2}}{3} ) ( Guinier ' s plot ) .$$

FIG. 20 shows non-limiting illustrative representations of flowerlike micelles of the sort formed by amphiphilic triblock copolymers in aqueous solution. R-lactide series polymers can form micelles with amorphous cores (left), whereas L-lactide series polymers can form micelles with crystalline cores (right).

FIG. 21A shows the structure of sulindac.

FIG. 21B shows the structure of tetracaine.

FIG. 22 is a graphical representation showing release of sulindac from polymer solution in PBS at 37° C.

FIG. 23 is a graphical representation showing release of tetracaine from polymer solution in PBS at 37° C.

FIG. 24 is a graphical representation showing release of tetracaine from polymer solution in nanopure water at 25° C.

FIG. 25 provides a representation of the distribution of drug in the PLA core for L-lactide series (A) polymers and R-lactide series polymers (B); L-lactide series polymers release drug much faster as the drug may be located on the periphery of the core because of high degree of packing inside the core. For R-lactide series polymers the drug can penetrate inside the core and its release is slower.

FIG. 26 provides a representation of release of drug from the micellar core. The shaded region represents the region of the core containing the drug. The drug is released at the boundary of the shaded region and it then diffuses out of the polymer matrix into water. It is assumed under this non-limiting model that the drug is uniformly dispersed in the PLA core (A) for R-lactide series polymers, whereas it lies on the boundary of the core (B) for L-lactide series polymers.

FIG. 27A graphically represents fits of equation 7 to the data for sulindac release from polymer solution in PBS at 37° C.

FIG. 27B graphically represents fits of equation 7 to the data for tetracaine release from polymer solution in PBS at 37° C.

FIG. 27C graphically represents fits of equation 7 to the data for tetracaine release from polymer solution in nanopure water at 25° C.

FIG. 28 graphically represents fits of equation 10 to the data for tetracaine release from polymer solution in PBS at 37° C.

### DETAILED DESCRIPTION OF CERTAIN EMBODIMENTS

The structure and properties of the copolymers of this invention, in a gel or hydrated state, can be characterized with one or more techniques specifically directed to establish structure-property relationships. These techniques include X-ray, neutron, and light scattering, as well as mechanical rheology and surface plasmon resonance spectroscopy. Correlations are established between polymer composition, hydrogel structure, and mechanical properties, and the evolution of mechanical properties over time is determined.

The process of gel formation, gel structure, and evolution over time are examined using physical methods including dynamic light scattering (DLS), Small Angle Neutron Scattering (SANS), wide angle X-ray diffraction (WAXD), Raman spectroscopy, ultra small angle neutron scattering (USANS), cryo-transmission electron microscopy (Cryo-TEM) and surface plasmon resonance spectroscopy (SPRS). These techniques allow characterization over multiple length scales from the atomic to bulk. Table 1 highlights the key experimental techniques, their length scale, the sample to be investigated, and the information available for each.

TABLE 1
Summary of techniques to characterize gel formation,
gel structure, and gel evolution.

| Technique | | | |
|---|---|---|---|
| Name | Length Scale | Sample Type | Data Available |
| DLS | 10-500 nm | dilute | CMC, micelle size, shape, |
| | | solution | population distribution |
| MP | n/a | dilute | CMC* |
| | | solution | |
| SANS | 1-100 nm | concentrated | PLA & PEO domain sizes, |
| | | to gel | micelle aggregation number, |
| | | | total micelle size, distance |
| | | | between micelles, hydration |
| | | | of PLA, interaction strength |
| | | | (χₐₛ) |
| USANS | 20-200 nm | concentrated | Total micelle size, large gel |
| | | to gel | structures, micelle ordering |
| WAXD | 0.1-1 nm | concentrated | LA crystallinity within |
| | | to gel | micelle |
| Raman | bond length | Gel | PLA conformations within |
| | | | micelle interior, micelle |
| | | | crystallinity |
| DSC | n/a | Gel | Micelle crystallinity, gel |
| | | | phase transitions |
| Cryo-TEM | 1-1000 nm | vitrified gel | Network structure, micelle |
| | | | size, distance between |
| | | | micelles |
| Rheology | Bulk | concentrated | Elastic & loss modulus, |
| | | to gel | strain & stress response, |
| | | | network lifetimes, complex & |
| | | | shear viscosity, strain |
| | | | hardening |
| SPRS | Bulk | Gel | Single phase or separated |
| | | | gel, dissolution rates |

*easier to obtain CMC from molecular probes (MP) than DLS; however, only gives concentration. This is a fast method to scan for CMC so that DLS instrument time is used more effectively.

DLS and SANS allow the monitoring of micelle size as the solution concentration of polymer is increased or decreased. Since micelle size is directly related to aggregation number, these two techniques provide a measure of any changes observed in aggregation number during gel formation or dissolution. The nature of the PLA domains within the hydrogel remains a central issue, and WAXD, Raman spectroscopy, and DSC methods are used to determine the state of the PLA micelles. USANS and cryo-TEM will provide structure information about the gel on a longer length scale. TEM can provide an image of the network structure even in the event that it is highly disordered and therefore does not produce a distinct scattering pattern. Mechanical rheology investigates the deformation behavior of the gel and SPRS will determine if the gels are a single phase or phase-separated, which significantly influences dissolution, drug release, and rheology properties. To summarize, these measurement techniques are specifically chosen to provide insight into gel structure and formation. The length scales provide overlap and permit the study of gel formation as it evolves from dilute solution.

Detailed structural information about PLA-PEO-PLA gels can also be obtained via SANS. The scattered intensity, I, is measured as a function of scattering vector, q. The advantage of SANS in examining polymeric systems is the ability to use a mixture of deuterated and hydrogenated solvents to contrast-match certain components and isolate scattering from either the PEO or PLA blocks. The q-range accessible through a conventional SANS experiment is approximately 0.003-0.3 Å⁻¹, corresponding to length scales on the order of 10-1000 A, ideal for micellar systems. SANS spectra from samples at various polymer concentrations and solvent mixtures are fit to existing models for polymeric micelles to obtain the size of PEO and PLA domains, the micelle aggregation number, the degree of hydration of the PEO and PLA domains, the total micelle size, and the mean distance between micelles in the gel state. Structure factor fits to these SANS data can also provide values for the intermicellar attraction, related to the interaction strength (χₐₛ) from associative network theories. These parameters are examined as a function of block copolymer composition and total molecular weight, as well as temperature, presence of PBS buffer or cell media, and presence of hydrophobic drugs or proteins for delivery applications. These molecular parameters can be used to tune the gel structure, and by comparison with rheology measurements, provide correlations between a bulk macroscopic property (elastic modulus), the system microstructure (micelle size, degree of hydration, etc.), and the chemical composition (PLA or PEO block length).

Ultra-small angle neutron scattering (USANS) can be used to probe larger length scales in the present hydrogels. Amphiphilic block polypeptides that form hydrogels are known to assemble into micron-scale structures, characterized by a power-law slope in the USANS q-range (0.0005-0.005 Å⁻¹). The mechanical properties of these polypetide gels are thought to be related to these large structures. In addition, this microstructure may be useful for tissue scaffolding applications. Previous work has shown that similar large-scale structures can also be formed in synthetic block copolymer hydrogels, with a corresponding power-law signature in the USANS spectra. USANS can be used to determine the extent to which formation of large-scale structures in PLA-PEO-PLA systems can be facilitated.

The structural nature of the hydrophobic micelles is a critically important question. Polymers with identical segments except for LA block crystallinity have been shown to have different bulk theological properties and micelle sizes. These techniques can be used to probe micelle environment, specifically looking for crystallinity in PLLA samples. Such techniques can be used to determine the degree of structural order present in the micelle interior, e.g., crystalline or amorphous polymer. For polymers composed of PLLA blocks, it is likely that micelles contain crystalline regions that can be observed by WAXD. Gels with high polymer concentration can be analyzed for crystallinity and compared with diffractograms obtained for the neat polymer. The influence of water on phase separation can be examined using high polymer concentrations to maximize the signal within the X-ray beam, since background scattering from water and random coil PEO blocks is present. For example, polymer C2-2 (see below) can be characterized in glass capillaries at 16 wt % polymer.

Raman spectroscopy (RS) can be used to characterize chain mobility. For gels that show diffraction, RS spectra are expected to show a carbonyl stretch around 1760 cm⁻¹ with both a higher and lower wavenumber shoulder. The region around 400 cm⁻¹ is expected to show two distinct bands for crystalline PLA and one broad band for amorphous PLA. The 700 cm⁻¹ band can be supportive of crystallinity. Alternatively, RS can be used to investigate PLLA and PDLLA blocks that form amorphous micelles because no signal will be present in WAXD. Amorphous PLA is characterized by a stronger band at 520 cm⁻¹, weak band at 411 cm⁻¹ and a higher population of tg⁻t conformers. The ability of aqueous solution, or gel formation, to enhance or diminish crystallization is important and a completely unanswered question in the literature. This is important because crystallization of PLLA domains significantly impacts degradation time and behavior. Crystallization is expected to influence the ability to release compounds in a controlled fashion, and can be shown using PLLA-PEO-PLLA copolymers with PLLA block lengths below and above the crystallization length. This block length is believed to be around 100 units based on DSC results. Further, the impact of micelle environment, whether crystalline or amorphous, on gel properties is believed to be significant, and correlated with mechanical rheology properties. DSC can be used to characterize the thermal properties of the copolymers, including PLLA Tₘ for the micelles, if present.

Direct imaging of hydrogel structure requires cryo-TEM techniques, which can provide excellent information on a hydrated organic sample. Samples are prepared from hydrogels with a range of moduli and crystallinity. For example, polymers that produce hydrogels with elastic moduli of 100, 1000, and 10000 Pa can be examined. For example, polymer C2-3 (see Table 3, below) with elastic modulus of 10 kPa described below can be examined at 5 different compositions ranging from just above the CMC to 16 wt % (10 kPa) to characterize the evolution of gel structure. In addition, two polymers of identical DP_{PEO}/DP_{PLA} can be examined in which one is composed of PLLA and the other of PDLLA.

Cryo-TEM sample preparation involves rapid freezing of hydrogel samples by immersion in liquid helium. This technique produces vitrified water that preserves the hydrogel structure. Samples are then fractured, placed on the instrument's cold finger, and visualized by TEM. Using this experimental technique, hydrogel structure can be observed and compared directly with SANS and DLS data collected.

Dynamic and steady rheological measurements are performed on bulk hydrogels using a stress-controlled Bohlin CVO rheometer and a strain-controlled Rheometrics ARES rheometer, utilizing either a cone-and-plate or cup-and-bob geometry, depending on the sample viscosity. These measurements yield relevant mechanical and viscoelastic properties, including elastic modulus (G′), loss modulus (G″), complex viscosity (η*), and shear viscosity (η). Dynamic measurements are made over a frequency range of 0.01-100 Hz. G′, G″, and η* are measured at a fixed frequency under strains of 0.05-100%. For liquid samples, steady shear experiments are performed over a shear rate range of approximately 0.001-10 s⁻¹. The rheology of these gels is measured as a function of temperature, pH, ionic strength, and presence of cell media, to determine their robustness under physiological conditions and determine if external triggers can be used to induce gelation or trigger degelation.

It is believed that the hydrogel elastic modulus is a strong function of the hydrophobe length and degree of crystallinity. Rheology measurements go beyond a simple evaluation of the liquid-gel boundary as a function of copolymer composition and concentration, providing quantitative values for the mechanical properties and a more complete set of data for comparison to existing associative network theories. Parameters such as the interaction strength χₐₛ, the junction lifetime, the midblock and hydrophobe molecular weights, and the micellar aggregation number can be varied.

In addition to examining linear viscoelastic parameters (i.e., G′, low-shear viscosity, etc.), studies can probe a wide range of frequencies, strains, and shear rates, providing information regarding nonlinear behavior of the materials. Nonlinear rheology can be crucial for biological applications that require a specified viscosity or elastic modulus over a range of external conditions, or a dramatic change in the mechanical properties under a certain mechanical stimuli (e.g., sudden yielding to facilitate delivery of drugs or proteins, rapid stiffening under high strains, etc.). It is desirable to design PLA-PEO-PLA hydrogels with nonlinear mechanical properties that mimic those found in soft tissue. One notable nonlinear feature is strain-hardening, where the elastic modulus shows a marked increase at high strains. This behavior is exhibited by a variety of biopolymer gels including gelatin, keratin, filamin, and fibrin, but is not typically seen in gels of synthetic block copolymers. This feature is believed to be due in part to the rigidity of microdomains within these gels, which can be reproduced by varying the crystallinity of the PLA domains.

SPRS can provide data regarding polymer and hydrogel structure as well as erosion. The mechanism of hydrogel dissolution is critical to both drug delivery and tissue engineering applications since these depend on solubility, degradation, and mechanical properties. Typically, hydrogels dissolve by one of two mechanisms including bulk swelling or slow surface erosion leading to dissolution. In the case of surface erosion, there is phase separation between a polymer dense gel and dilute sol phase. Preliminary investigation on polymers C2-2 and C2-3 (see below) showed phase separation in these systems. Phase separated hydrogels can to be adapted to drug delivery applications since the properties change in a predictable manner.

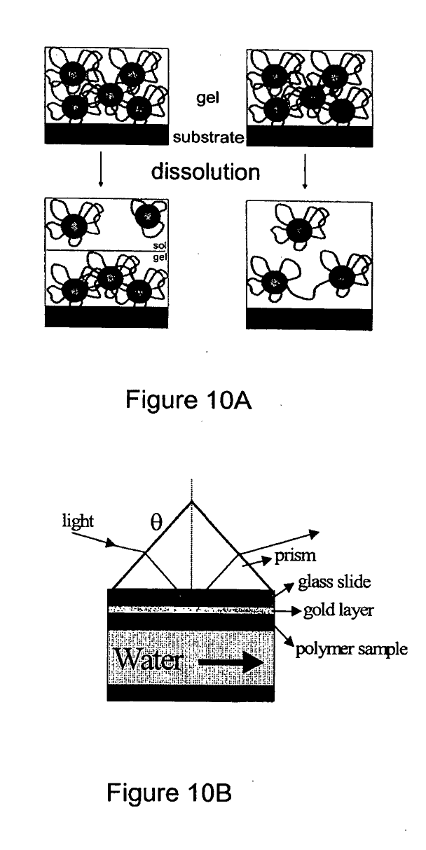

SPRS is a newer technique that has been used to investigate surface erosion of fluoroalkane end block modified PEGs. This technique works extremely well when polymer dissolution occurs over several hours, which is common for samples showing phase separation as observed for polymers C2-2 and C2-3. SPRS relies on the fact that a thin gold film's plasmon resonance angle is very sensitive to the thickness and refractive index of any material in contact with it. (See, e.g., FIG. 10B.) The thickness limitation is ˜1 μm. Polymer samples will be prepared, mounted and monitored as water or phosphate buffer solution (PBS) solution is passed over the polymer gel film. Polymer films are prepared by spin-casting from chloroform and thickness controlled by polymer concentration of the solution. Two film thicknesses of approximately 0.5 and 2 μm are prepared and their thickness is confirmed by ellipsometry.

For the thinner samples, a rapid change in signal is expected as the polymer is hydrated and swollen with water resulting in a thickness and refractive index change. After this initial change, the response is expected to be constant for a given time period that is linearly related to film thickness as the polymer dissolves from the water-polymer interface but is not detected because the interface is more than 1 μm from the gold surface due to polymer swelling. Finally, as the swollen film thick decreases into the detection level, a steady decrease in angle should be observed. Thicker films are expected to show a smaller initial signal change (since film thickness is already greater than 1 μm) as well as a longer constant angle regime. The two samples can be used to probe differences in swelling during the initial period and will correlate film thickness with the constant signal region of the experiment. In contrast, if the polymers exhibit bulk swelling then a constant change in signal over the entire experiment is expected as the refractive index continually evolves during polymer dissolution.

Ring opening polymerization techniques have been used to prepare a series of triblock copolymers composed of PEO mid-blocks and varying length LLA end segments as described in Table 2. Since these polymers contain large hydrophilic PEO mid-blocks, the materials swell dramatically in aqueous solution and form hydrogels at relatively low concentrations (>20 wt %). Table 2 summarizes molecular weight features of exemplary series that incorporate PEO segments of 8,900 MW.

In one embodiment using L-lactide—the cyclic dimer of LLA, stannous-2-ethyl hexanoate is employed as catalyst for the ring opening polymerization as shown in Scheme 1, below, and more fully in example 1a. (PDLA can be prepared, analogously, from PEO and dimeric DLLA.)

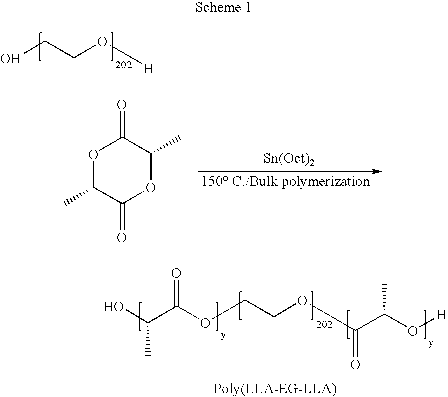

Stannous-2-ethyl hexanoate [Sn(C₈H₁₅O₂)₂] is also known as stannous-2-ethylhexoate, stannous octoate, Sn(Oct)₂ and tin octoate. Stannous-2-ethyl hexanoate is a well characterized catalyst for the ring opening polymerization and easy to handle in the laboratory. In alternative embodiments, catalysts such as Zn or Na metal and potassium t-butoxide can be used, especially if residual catalyst is a toxicity concern.

The first chemical composition variable explored was the hydrophobic block length as shown in the table. Using PLLA is preferable for this purpose because the block is semi-crystalline and will more strongly favor microphase separation compared PDLLA mixtures. Table 2 shows the molecular characteristics for 11 polymers that were prepared. The DP for LA is based on ¹H-NMR integration since common standards for gel permeation chromatography (GPC) do not exist for these copolymers. In addition, polymers 3, 4, and 6, which are highlighted in Table 2, were prepared in >10 g quantities so that all physical property studies could be performed on the same polymer batch. Larger quantity polymerizations can be performed without difficulty.

The fundamental nature of polymerization produces a different composition of chain length populations on each occasion. While this is unavoidable, carefully chosen molecular architecture, as in this case, can minimize the number of variables that can change between polymer batches. In this case, chain length is the only variable, and side reactions are negligible under the reaction conditions. In addition, the catalyst was specifically chosen to prevent racemization of the chiral center. These properties are in contrast to many other potential polymers and polymerization conditions in which the stereocenter is racemized or newly created with little control. Examples include the polymerization of styrene, isoprene, butadiene, acrylates, and propylene in which batch-to-batch polymerization can never reproduce the exact same molecular chain stereochemistry. All polymers in Table 2 were characterized by ¹H- and ¹³C-NMR, GPC, elemental analysis, and MALDI-TOF mass spectrometry.

TABLE 2
Molecular weight characteristics of PLA-PEO-PLA triblock copolymers.

| Sam- | | | | Total | Wt | | DP_{PEO}/ |
|---|---|---|---|---|---|---|---|
| ple | M_{nPEG}^{a} | M_{nPLA}^{b} | M_{Total}^{c} | DP_{PLA}^{b} | %_{PEO} | WT %_{PLA} | DP_{PLA} |
| 1 | 8,900 | 1,872 | 10,772 | 26 | 82.6 | 17.4 | 7.8 |
| 2 | 8,900 | 2,232 | 11,132 | 31 | 79.8 | 20.2 | 6.5 |
| 3 | 8,900 | 3,456 | 12,356 | 48 | 72.0 | 28.0 | 4.2 |
| 4 | 8,900 | 4,320 | 13,220 | 60 | 67.3 | 32.7 | 3.4 |
| 5 | 8,900 | 4,752 | 13,652 | 66 | 65.2 | 34.8 | 3.1 |
| 6 | 8,900 | 5,184 | 14,084 | 72 | 63.2 | 36.8 | 2.8 |
| 7 | 8,900 | 5,328 | 14,228 | 74 | 62.5 | 37.5 | 2.7 |
| 8 | 8,900 | 6,192 | 15,092 | 86 | 59.0 | 41.0 | 2.3 |
| 9 | 8,900 | 8,640 | 17,540 | 120 | 50.7 | 49.3 | 1.7 |
| 10 | 8,900 | 11,520 | 20,420 | 160 | 43.6 | 56.4 | 1.3 |
| 11 | 8,900 | 12,240 | 21,140 | 170 | 42.1 | 57.9 | 1.2 |

^{a}Determined by SEC;
^{b}Determined by ¹H NMR;
^{c}Polydispersity Index averages 1.2.

Gels were prepared by slow addition of an aliquot of dried polymer to a fixed volume of deionized water (DI) water (typically 2 or 15 mL). Table 3 summarizes the polymers used and wt % concentration of the polymer of the prepared gels. The solution is stirred and heated periodically at 40 degrees Celsius until a homogenous phase is formed. In all cases, except for polymer C2-0, polymer was added until a hard gel was formed. A hard gel was operationally defined by immobilization of the stir bar and no observed flow over a period of 5 min. In other embodiments, dried polymer is added until a viscous solution is formed which remains at the bottom of the vial for >2 min after inversion. Gels were then transferred to the Bohlin CVO rheometer and measurements performed using a cone-and-plate geometry with a 4° cone, 40 mm diameter plate, and 150 μm gap. (See, examples, below.)

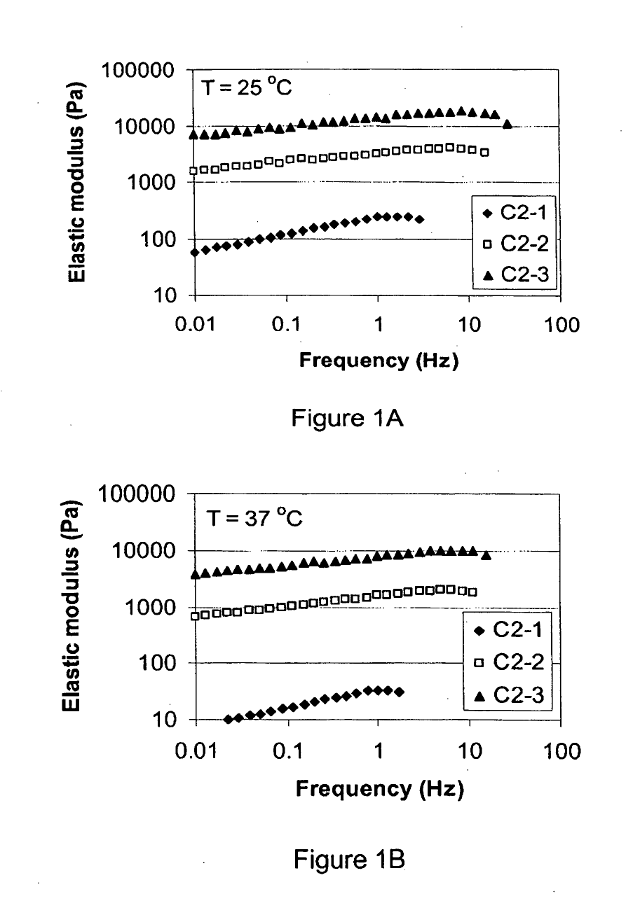

All samples were characterized by dynamic mechanical rheology, except for example C2-0, which did not form a gel up to 20 wt %. FIG. 1A shows the elastic modulus (G′) response vs. frequency (Hz) at 25° C. of the three gels formed by polymers C2-1, C2-2, and C2-3. A resonance frequency of the rheometer motor at high Hz limited the high-frequency range for these data sets. Strong gels were formed with polymers C2-1, C2-2, and C2-3 at 20, 16, and 16 wt %, respectively. Three observations can be made regarding the curves in FIG. 1A. First, although the gel of polymer C2-1 was prepared in 20 % compared to 16 wt % for the other materials, it forms a weaker gel. Second, polymer C2-3 forms a strong gel with G′≧10,000 Pa and, third, the hydrophobic length or DP has a pronounced effect on elastic modulus. The only difference between these polymers is the total DP of the PLLA segments, which are 48, 60, and 72, respectively.

TABLE 3
Samples for preliminary rheology experiments.

| | | DP_{PEO} | | DP_{PLLA} | Wt % |
|---|---|---|---|---|---|
| Sample | MW_{PEO} | (total) | MW_{PLLA} | (total) | polymer |
| C2-0 | 8,900 | 202 | 1,872 | 26 | 20.0 |
| C2-1 | 8,900 | 202 | 3,456 | 48 | 20.0 |
| C2-2 | 8,900 | 202 | 4,320 | 60 | 16.0 |
| C2-3 | 8,900 | 202 | 5,184 | 72 | 16.0 |

As the length of the hydrophobic block is increased from 24 (half of 48 since there are two PLLA ends) to >30, gel strength improves by more than two orders of 20 magnitude from ˜100 Pa to 10,000 Pa even though the % of polymer in the gel is decreased. These observations demonstrate the effect that hydrophobic domains can have on mechanical properties and supports the contention that control over the hydrophobic block length can be used to control gel strength.

FIG. 1B shows G′ vs. frequency (Hz) at 37° C. with a decrease in G′ for all three gels but a much larger decrease for C2-1 than either C2-2 or C2-3. This decrease in G′ with increasing temperature is common for gels composed of PLA-PEO-PLA molecules. The relative lack of dependence on frequency is expected due to the ‘hard’ nature of these gels, and indicates that regardless of the end application, the mechanical environment will have little influence on the polymeric device's performance.

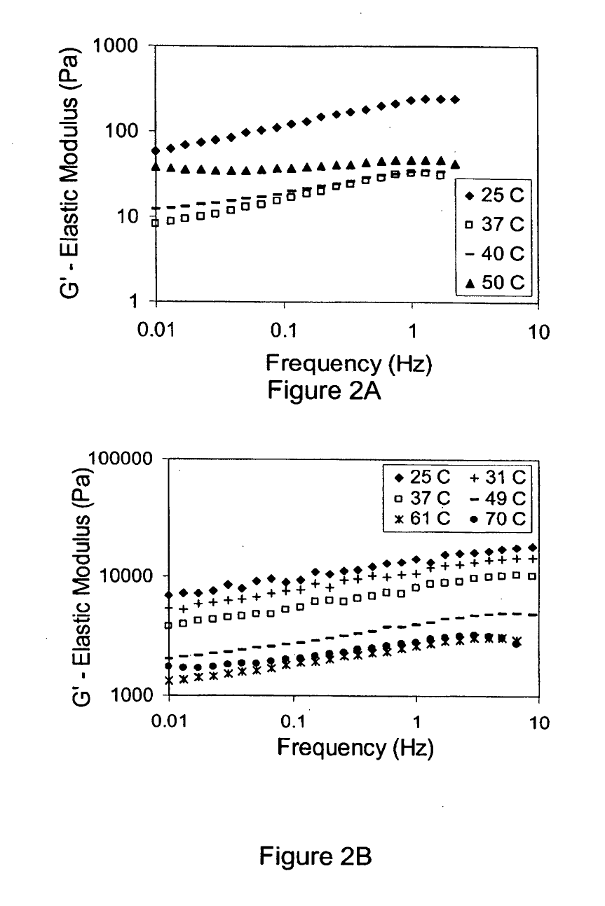

FIGS. 2A and 2B show the influence of temperature on G′ for samples C2-1 and C2-3, respectively. FIG. 2A is a graphical representation of the temperature dependence of elastic modulus of hydrogels formed from sample C2-1. G′ for sample C2-1 decreases with temperature as expected near 40° C. the sample undergoes a transition toward increasing G′. At 50° C., the elastic modulus is even more linear with temperature and is significantly higher at low frequencies as compared to the results obtained at 37° C.

While not to be held to a particular hypothesis, it is believed that at 25° C., the hydrogel is composed of solid or slightly hydrated micelles but as the temperature is increased near 40° C., these micelles begin to melt and destabilize (T_{g} of PLLA is around 50° C. and can be lowered by the presence of water as a weak plasticizer). Then, as the temperature is increased further, the PLA domains can dehydrate in a manner similar to that observed for the PPO segments of Pluronics®. In contrast, sample C2-3 behaved in a more expected manner with a steady decrease in G′ as the temperature is increased. FIG. 2B is a graphical representation of the temperature dependence of elastic modulus of hydrogels formed from sample C2-3. The 70° C. data shows a slight increase in modulus, which would be expected since water is less likely to hydrate larger PLLA blocks. The overall decrease in modulus is significant, but not dramatic enough that a sol-gel transition is observed.

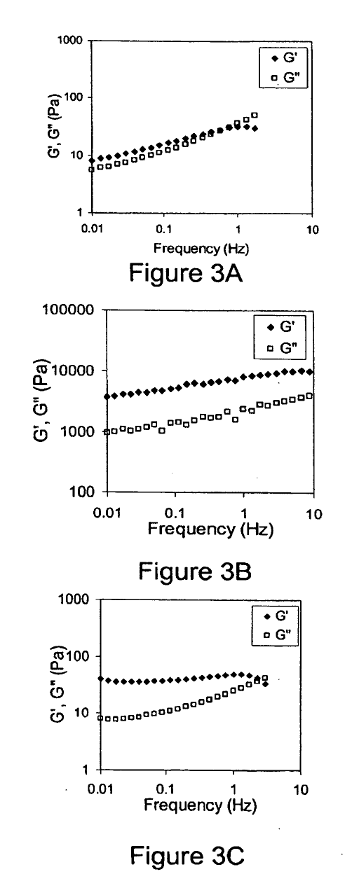

Sol-gel transitions are defined as G′>G″ and FIG. 3A shows that for sample C2-1 at 37° C. G′≈G″ for the entire range of frequency. FIG. 3A is a graphical representation of the viscoelastic moduli for hydrogels formed from sample C2-1, T=37° C. In contrast, G′ is always greater than G″ for C2-3 regardless of temperature (up to 70° C.), as shown in FIG. 3B for data at 37° C. FIG. 3B is a graphical representation of the viscoelastic moduli for hydrogels formed from sample C2-3, T=37° C. The increase in G′ at 50° C. for sample C2-1 is also seen in FIG. 3C where G′ is much larger than G″. FIG. 3C is a graphical representation of the viscoelastic moduli for hydrogels formed from sample C2-1, T=50° C., showing gelation (G′>G″). The frequency-dependent G′ observed for sample C2-1 at 25° C. and 37° C., as opposed to the frequency-independent behavior observed at 50° C., can indicate the presence of a slow relaxation mechanism at lower temperatures.

The temperature range extended up to 70° C. for polymer C2-3, and no clear sol-gel transition in DI water was observed for biologically relevant temperatures (25-45° C.). In other embodiments, PBS, other growth media, or tetraglycols can affect the gel transitions, as well as the influence of weight % composition. It is expected that a more dilute solution (˜12 wt %) can provide a sol-gel transition near physiological temperatures without a dramatic decrease in modulus.

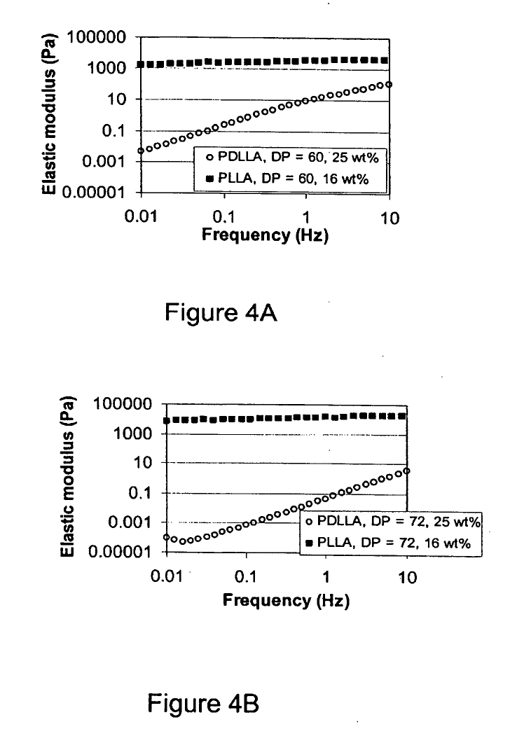

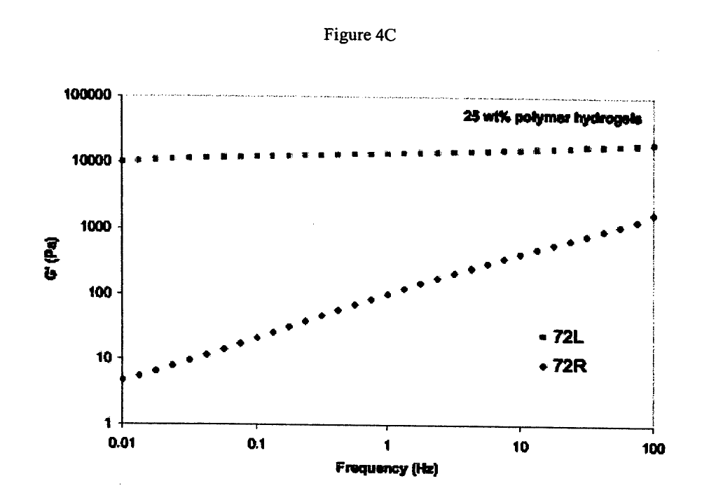

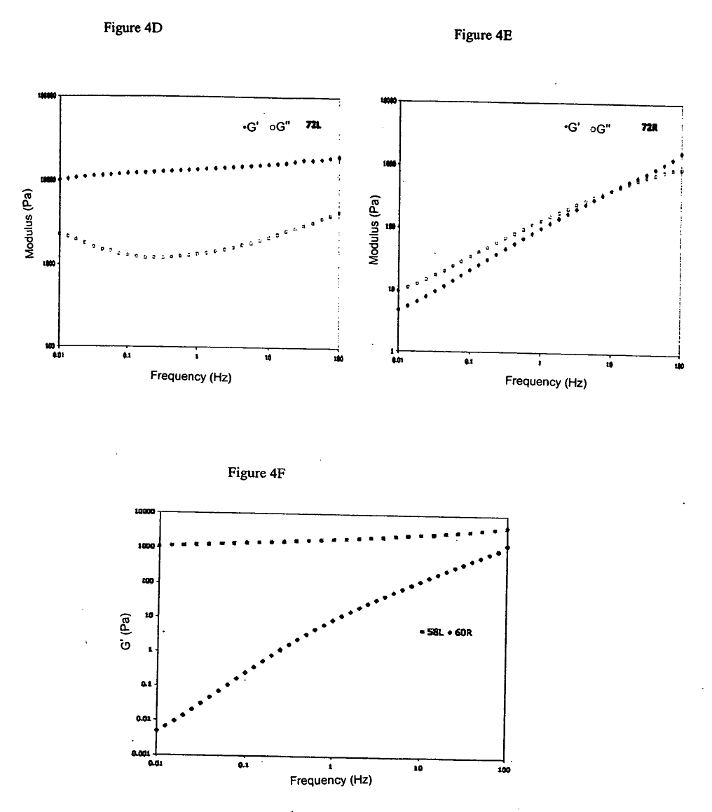

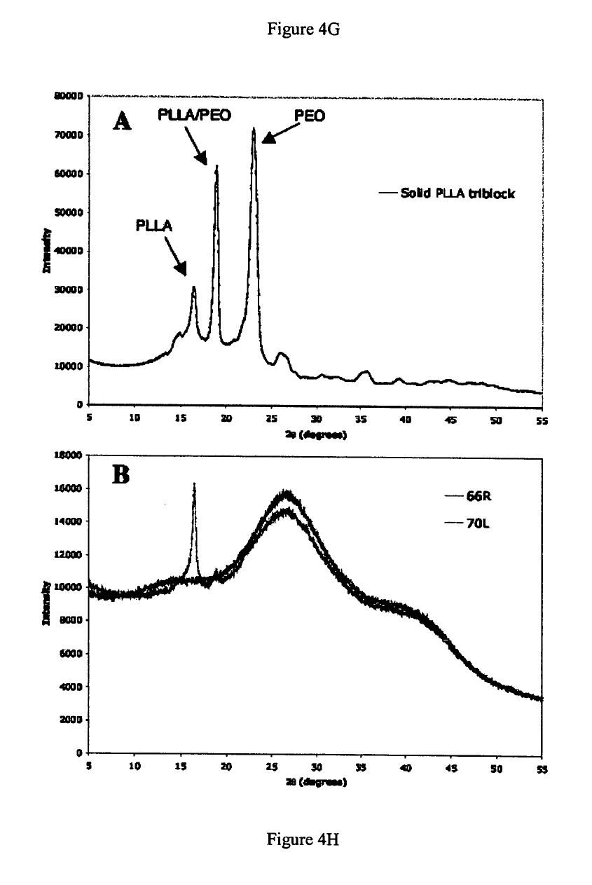

PLLA was specifically chosen to test whether crystalline hydrophobic domains will lead to gels with a higher modulus. Blocks of PLLA are known to crystallize while blocks composed of PDLLA are amorphous. Polymer samples were prepared from identical PEO mid-blocks and either 60 or 72 LA units. However, one set of polymers was composed of LLA while the other set contained DLLA. Gels were prepared from these samples, as described above, and the mechanical rheology studied. The elastic moduli are compared in FIGS. 4A and 4B, and in Table 4, below. FIG. 4A is a graphical representation of the elastic modulus for hydrogels formed from triblocks with PDLLA and PLLA endblocks, with the same DP of PLA: 60 units. FIG. 4B is a graphical representation of the elastic modulus for hydrogels formed from triblocks with PDLLA and PLLA endblocks, with the same DP of PLA: 72 units.

TABLE 4
Elastic Modulus At Representative Frequencies

| | Elastic Modulus (Pa) | | | |
|---|---|---|---|---|
| | PDLLA, | PLLA, | PDLLA, | PLLA, |
| | DP = 60, | DP = 60, | DP = 72, | DP = 72, |
| Frequency (Hz) | 25 wt % | 16 wt % | 25 wt % | 16 wt % |
| 0.01 | 6.9 × 10⁻³ | 1,350 | 1.1 × 10⁻⁴ | 6,210 |
| 0.1 | 0.28 | 2,100 | 9.2 × 10⁻⁴ | 10,000 |
| 1 | 7.4 | 3,280 | 0.049 | 13,740 |
| 10 | 108 | 3,280 | 5,184 | 16,100 |

Although the PDLLA (i.e., R, in FIGS. 4C-F) samples are at a higher concentration than the PLLA (i.e., L, in FIGS. 4C-F) samples, their moduli are still orders of magnitude lower than the moduli of the PLLA-based gels. These data indicate that hydrophobe crystallinity, which can be controlled by the microstructure of the PLA block, has a dramatic effect on the elastic modulus.

As an extension of the foregoing, comparable PLA-PEO-PLA triblock copolymers were prepared using both L lactide and DL-lactide (or “R”) monomer. With chain lengths and molecular weights on the order described above, these polymers were dissolved in water at concentrations ranging from 10-25 wt % polymer, and they were qualitatively divided into two categories: (1) those that pass the vial-inversion test and (2) those that fail. In all cases, the gels containing PRLA failed the vial inversion test, while gels composed of PLLA passed when the concentration exceeded approximately 16 wt %. To better quantify the gel strength, rheological measurements were performed with a cone and plate geometry that allows the viscoelastic moduli versus frequency to be measured. This study also allows a direct comparison between stereoregular and stereorandom polymers.

Again, the storage modulus was found to be strongly dependent on the stereochemistry of the PLA blocks. When hydrogels were made from polymers with identical degrees of polymerization and at the same concentration, the gels composed of PLLA blocks were significantly stiffer than those containing racemic PLA blocks. A typical example is shown in FIG. 4C where a stereoregular sample, DPLA=72, forms a stiff gel while the racemic sample does not. For example, at 1 Hz the stereoregular gel has a storage modulus of 14 kPa while the racemic solution's storage modulus is 0.1 kPa. The gel formed by stereoregular 72L displays an elastic modulus that is independent of frequency as expected for a hard gel while the much softer racemic sample displays the expected frequency dependence.

The increased stiffness was thought due to the formation of hydrophobic domains in which the PLLA blocks crystallize in the core while the racemic polymer forms a classic micelle because the hydrophobic domains cannot crystallize due to the stereorandom structure. This hypothesis is further supported by the Wide Angle X-Ray Diffraction (WAXD) data shown in FIGS. 4G-H. The peaks in the powder sample (FIG. 4G) at 2θ≈19° and 23° are characteristic of crystalline PEO. PLLA is also known to exhibit peaks at 2θ≈19° and 22°, so there is overlap between the crystalline PEO and PLLA peaks; however, the peak at 2θ17° is due solely to crystalline PLLA. This confirms the presence of crystalline PLLA and PEO in neat solid samples of these triblock copolymers. When the sample is hydrated to form the gel, the peaks corresponding to crystalline PEO disappear while those from PLLA remain and appear to sharpen upon gel formation. This confirms that the PLLA domains are crystalline in the gel sample and may be more well ordered than in the powder form. Meanwhile, the sample formed from racemic PLA shows no peaks corresponding to crystalline PLA as expected. These WAXD studies confirm the hypothesis that crystalline PLA segments are present in the gels formed by the stereoregular triblock copolymer and that crystallinity of these blocks likely acts to stabilize the hydrophobic domains, resulting in a stiffer hydrogel.

Accordingly, the stiffness of hydrogels made from PLA-PEO-PLA can be controlled by the stereoregularity of the polymer. This increased stiffness appears to be related to the formation of crystalline hydrophobic PLLA blocks in the gel as evidenced by WAXD. Gels with PLLA hydrophobic domains are considerably stiffer than those with PRLA hydrophobic blocks of identical length. The use of PLA allows a simple chemical change, stereochemistry, to be altered while holding all other molecular parameters constant, thus allowing the impact of stereochemistry to be measured directly. This tunability can be used designing hydrogels and, for the first time, crystallinity is shown to influence gel strength directly.

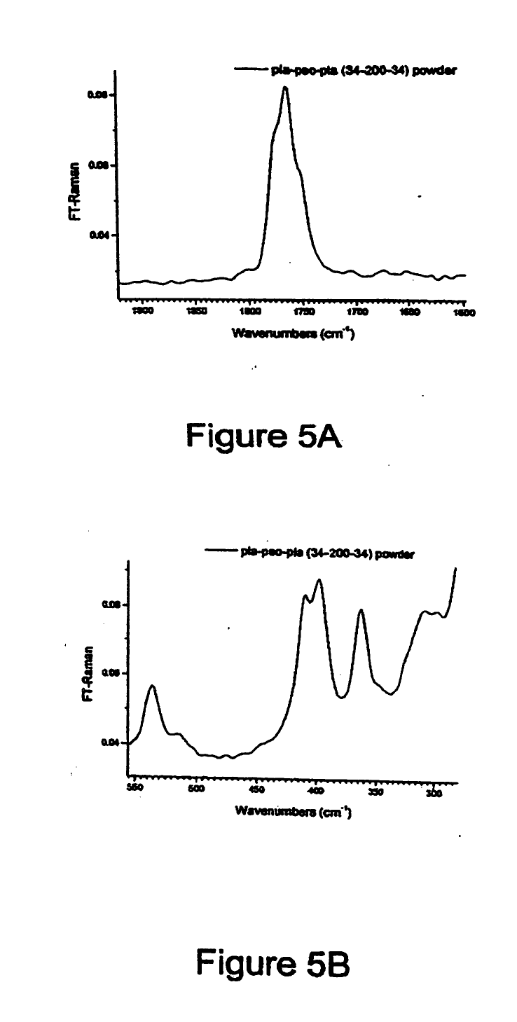

The influence of crystallinity on hydrogel modulus is also supported by the results of Raman spectroscopy when used to evaluate PLLA crystallinity in the gels. The results are shown in FIGS. 5A and 5B for the carbonyl and lower wavenumber region. FIG. 5A is a Raman spectrum of a PLLA gel showing the carbonyl stretch; FIG. 5B is a Raman spectrum of PLLA gel showing the bending region. FIG. 5A shows the carbonyl stretch has both a higher and lower wavenumber shoulder on the main band which is strong evidence for crystallinity. In agreement, the bands at 398 cm⁻¹ and 417 cm⁻¹ are known to split into two distinct peaks early in the crystallization process. These results strongly support crystallinity in the PLLA gels. In contrast, the results of similar Raman spectroscopy studies on PDLLA gels are consistent with amorphous PLA.

The structure-property relationships of elastic moduli of PLA-PEO-PLA gels and to PLA block length and crystallinity was explored using dynamic light scattering (DLS) and small-angle neutron scattering (SANS) on a series of solutions and gels with varying PLA block length and crystallinity (Table 5).

Table 4 presents the characteristics for polymers used for DLS and SANS experiments, along with major results from DLS. Samples for DLS were prepared by dissolving the polymer in filtered, deionized water (Nanopure) followed by stirring, heating, and subsequent filtration through 0.22 micron syringe filters. For each polymer, several samples were prepared to cover the concentration range of 0.001-0.2 wt %. Experiments were performed at 25° C. using an Ar laser at a wavelength of 514 nm with a Brookhaven BI-9000 correlator. DLS showed spherical micelles for all samples. Micellar sizes, expressed as the hydrodynamic radius R_{H}, were in the range 29-80 nm. It can be seen that R_{H} increases within each series of increasing PLA block length. A comparison of data for samples 2L and 2D/L, and 3L and 3D/L, shows that the micellar sizes for the PLLA-based copolymers are consistently higher than those for the PDLLA based copolymers.

TABLE 5
Characteristics of PLA-PEO-PLA triblock copolymers for SANS and
DLS studies, and micellar parameters from DLS.

| | | | | CMC, | R_{H} at | Wt % polymer for SANS |
|---|---|---|---|---|---|---|
| Sample | M_{nPEG} | M_{nPLA} | Total DP_{PLA}^{b} | wt % | CMC, nm | (behavior at 25° C.) |
| 1L | 8,900 | 1,872 | 26 (PLLA) | 0.005 | 64.8 | 0.5 (sol), 10.0 (sol), 22.0 |
| | | | | | | (sol) |
| 2L | 8,900 | 4,320 | 60 (PLLA) | 0.001 | 69.4 | 0.5 (sol), 10.0 (sol), 22.0 |
| | | | | | | (gel) |
| 3L | 8,900 | 5,184 | 72 (PLLA) | 0.0025 | 79.5 | 0.5 (sol), 10.0 (sol), 22.0 |
| | | | | | | (gel) |
| 2D/L | 8,900 | 4,320 | 60 (PDLLA) | 0.005 | 29.8 | — |
| 3D/L | 8,900 | 5,184 | 72 (PDLLA) | 0.005 | 33.8 | — |
| 4D/L | 8,900 | 6,336 | 88 (PDLLA) | 0.005 | 43.6 | — |

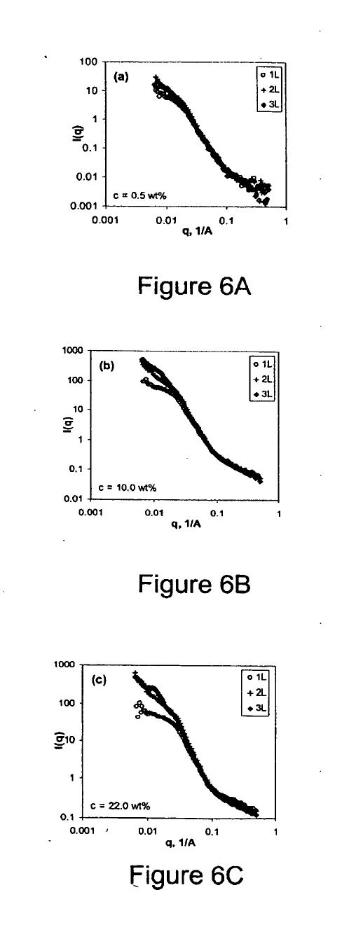

SANS was also performed on a series of solutions and gels with increasing PLLA block length (samples 1L, 2L, and 3L, Table 5). Samples were prepared by addition of dried polymer to D₂0, followed by stirring and heating. SANS was carried out on the small-angle diffractometer at the Intense Pulsed Neutron Source, Argonne National Laboratory. Data were obtained at 25° C. for 0.006 ÅA⁻¹>q>0.5 Å⁻¹, corresponding to length scales of 1.2-3100 nm. Spectra shown in FIGS. 6A-6C indicate that at concentrations of 0.5 wt % and above, these triblocks do not form “flowerlike” spherical micelles, as commonly believed. FIG. 6A is a graphical representation of results of small angle neutron scattering (SANS) tests showing spectra at 25° C. for PLLA-PEO-PLLA solutions and gels with increasing PLLA block length at a polymer concentration of 0.5 wt %. FIG. 6B is a graphical representation of results of small angle neutron scattering (SANS) tests showing spectra at 25° C. for PLLA-PEO-PLLA solutions and gels with increasing PLLA block length at a polymer concentration of 10.0 wt %. FIG. 6C is a graphical representation of results of small angle neutron scattering (SANS) tests showing spectra at 25° C. for PLLA-PEO-PLLA solutions and gels with increasing PLLA block length at a polymer concentration of 22.0 wt %.

The data are consistent with formation of aggregates that are polydisperse and ill-defined; moreover, the size and shape of the aggregates does not appear to depend strongly on PLLA block length. However, PLLA block length does impact association or bridging between aggregates, as shown in FIG. 6B and FIG. 6C. The spectra from the two gel-forming samples show an increase and change of slope at low q, indicating the presence of large-scale structure in these systems, presumably due to association or network formation. Larger PLLA block lengths also lead to weak ordering of the aggregates, as seen in FIG. 6C. At the highest concentration, a peak at q=0.0117 Å⁻¹ in the sample with the largest PLLA block, was observed corresponding to an interaggregate spacing of about 53.6 nm. Thus, the degree of aggregate association and ordering has a large effect on the macroscopic rheology. It is also expected that ordering and association also similarly affect the impact mass transport and release characteristics of these gels.

As an extension of this work, a complete structure characterization of PLA-PEO-PLA triblock copolymers was performed in aqueous solution and gel state, to compare the structure of PLA-PEO-PLA polymers made from an amorphous racemic mixture of D and L lactic acid blocks against that made from crystalline L-lactic acid blocks. With reference to the following examples, the two series of polymers are seen to self-assemble into significantly different structures in solution and gel as is represented by differences in their SANS spectra. The racemic lactide series polymers (i.e., PDLLA or “R”) are seen under the conditions employed to form flower-like micelles in dilute solutions. With an increase in concentration of the polymer in solution, the hydrophobic end groups associate with the neighbouring micelles to form a highly ordered network of spherical micelles. Such associations are theoretically expected and are believed, without limitation, to occur due to the ability of the small hydrophobic end groups to form bridges with the adjacent micelle. The crystalline L-lactide polymers on the other hand form polydispersed micelles, which would be similar in structure to lamellar micelles. Because of their random orientation and polydispersity in their size these polymers form a randomly crosslinked, polydispersed network structure at higher concentrations. Data obtained from fits of physical models to the experimental data shows that the radius of spherical micelles formed for R-lactide polymers lies in the range 9-11 nm, whereas size of inhomogenities in L-lactide polymers lies in the range 7-15 nm. Thus despite significant differences in their structure, the size of the moieties formed in the two series of polymers is comparable. The association properties of the polymer aggregates in solution are observed to be dependent upon the length and crystallinity of the hydrophobic PLA block. The changes in structure and association behavior of the gels upon changes in chemistry of the PLA block can be related to macroscopic properties of the gels of these polymers in aqueous solution.

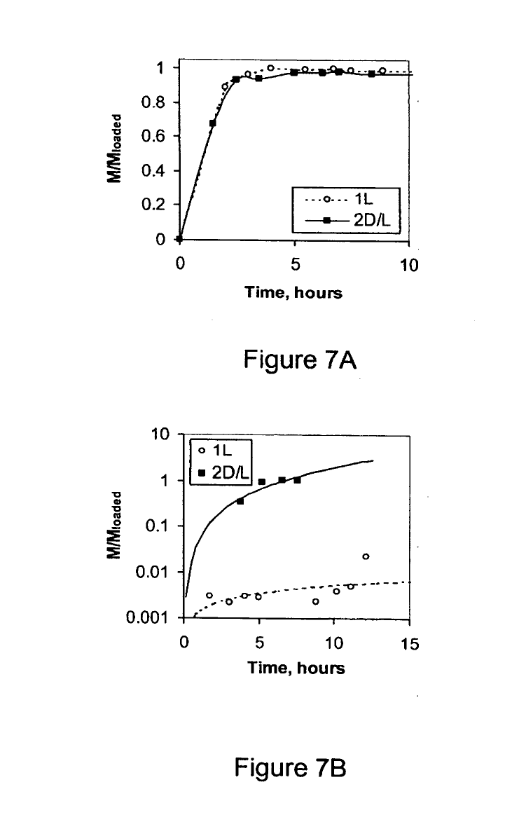

The use of thermo-reversible triblock gels (Pluronics) as delivery agents for anti-inflammatories (naproxen and indomethacin), local anesthetics, and anti-cancer agents has been studied (Sharma, P. K. and Bhatia, S. R. Effect of Anti-Inflammatories on Pluronic® F127: Micellar Assembly, Gelation and Partitioning. Int J Pharmaceutics, in press, 2004). An ultraviolet spectroscopy technique has been developed to measure rapidly the solubility and micelle-water partition coefficients of hydrophobic pharmaceuticals in micellar gels and has been applied to PLA-PEO-PLA hydrogels. In addition to evaluating partitioning, release kinetics in PLA-PEO-PLA systems have also been studied. Preliminary data on release kinetics at 25° C. are shown for two pharmaceuticals, methyl paraben (FIG. 7A) and indomethacin (FIG. 7B) for the triblock polymers IL and 2D/L from Table 5. FIG. 7A is a graphical representation of the release profile methyl paraben from PLA-PEO-PLA gels, loaded in systems with PLLA endblocks (IL) and PDLLA endblocks (2D/L). FIG. 7B is a graphical representation of the release profile indomethacin from PLA-PEO-PLA gels, loaded in systems with PLLA endblocks (1L) and PDLLA endblocks (2D/L).

Indomethacin is relatively more hydrophobic than methyl paraben, with a solubility in water of 25 μg/mL and a micelle-water partition coefficient of 108±8. This indicates that indomethacin is residing primarily in the hydrophobic PLA regions. By contrast, methyl paraben is more hydrophilic, with a solubility of 2360 μg/ml. FIG. 7A shows the amount of methyl paraben released over time, normalized by the amount initially loaded into the polymer. The profiles are similar for both polymers, with the majority of the methyl paraben being quickly released within the first five hours.

FIG. 7B shows that the release is quite different for indomethacin. For sample 2D/L, which has amorphous hydrophobic domains, release is still fairly rapid, although slower than is observed for methyl paraben. However, release from sample 1L, which has PLLA-based hydrophobic domains, is relatively delayed, with reservoir-like release over a period of several hours, indicating that complete release (M/M_{loaded}=1) would occur on the order of days for this system. Thus, the release profile and rate of hydrophobic pharmaceuticals is strongly dependent on the chemistry of the PLA block and the crystallinity of the hydrophobic domains. It is believed that the differences between methyl paraben and indomethacin are due to their differing solubilities and location within the gel structure (i.e., partitioning into PLA domains versus location in hydrophilic domains). Thus, release characteristics for a specific drug can be tuned by making the appropriate adjustments in the block structure of the polymer.

Based on the foregoing, continued synthesis and testing of triblock copolymers with variable PLLA provides additional materials with tailored properties, such as improved depot characteristics for hydrophobic drugs. Consider use of the copolymers of this invention with two such representative agents.

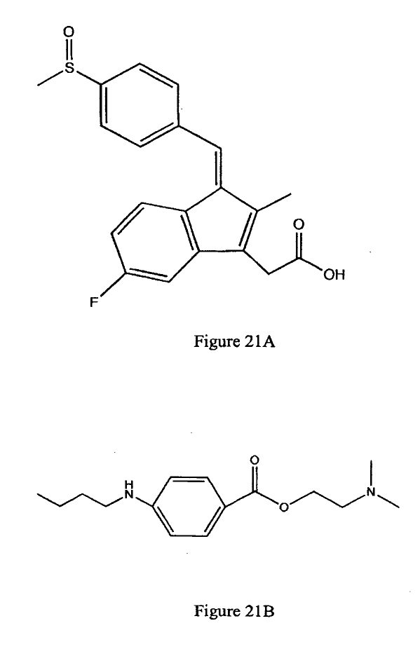

Sulindac and tetracaine are primarily hydrophobic with very low water solubility (see structures in FIGS. 21A-B). Researchers have described previously the use of poly(styrene-divinylbenzene) microspheres for the release of the anti-inflammatory drug sulindac, as well as poly(DL-lactic-co-glycolic acid) for release of tetracaine hydrochloride, which is a local anaesthetic. In the former case, complete release of sulindac was seen to occur over 24 hours whereas in the latter case a burst release of tetracaine hydrochloride was seen to precede sustained drug release behavior. In both the cases, continuous and prolonged drug release behavior was not achieved. With reference to the following examples, in contrast to the art, micellar solutions of PLA-PEO-PLA yield almost zero-order, prolonged and continuous release patterns of sulindac and tetracaine. The rate of drug release is significantly modified by even slight changes in the block composition and crystallinity of the block copolymer, and hence controlled release rates of the drugs can be achieved.

TABLE 6
Characteristics of PLA-PEO-PLA triblock
copolymers with variable PLLA lengths.

| MW_{PEG} | Total DP_{PLLA} | MW_{PLLA} | MW_{Total} |
|---|---|---|---|
| 8,900 | 10 | 720 | 9,620 |
| 8,900 | 16 | 1,152 | 10,052 |
| 8,900 | 20 | 1,440 | 10,340 |
| 8,900 | 200 | 14,400 | 23,300 |
| 8,900 | 300 | 21,600 | 30,500 |
| 8,900 | 400 | 28,800 | 37,700 |
| 8,900 | 800 | 57,600 | 66,500 |

Several PLLA block lengths have been prepared as described above in Table 3. Three of these have been screened for hydrogel formation and their mechanical properties determined in rheology studies (FIGS. 1A-3C). To determine the hydrophobic block length over which hydrogels can be formed, shorter and longer PLLA blocks are prepared as summarized in Table 6, above. Hydrogels can be formed even in copolymers with very large PLA blocks compared to PEO blocks, such as PLA₂₆₄-PEO₁₈₂-PLA₂₆₄ (Molina, I., et al. Protein Release from Physically Crosslinked Hydrogels of the PLA/PEO/PLA Triblock Copolymer-type. Biomaterials 22: 363-369, 2001). While total MW is not a concern since PLLA blocks are degradable, there may be an upper limit of LA block length for a given PEO mid-block that limits hydrogel formation. Such an upper limit has not been observed for DP_{PLLA} up to and including 170. Synthesis and testing of copolymers having DP_{PLLA} of about 200-800 demonstrate the role of this parameter in the formation of hydrogels.

Data presented above indicates that hydrophobe crystallinity influences overall mechanical properties as well as micelle size. In order to establish a structure-property relationship based on crystalline or amorphous PLA blocks, polymers are prepared with the same chemical composition and block lengths, but one polymer will contain solely LLA while the other is composed of an equal mixture of the L and D enantiomers. These polymers are prepared using the PEO segments already studied and described above, and have MW of 8,900. Six types of polymers are studied as outlined in Table 7, below.

TABLE 7
Two sets of polymers with identical chemical formula and composition
are prepared, differing only in the crystallinity of PLA blocks.

| | Total | |
|---|---|---|
| Hydrophobe | DP | Structure |
| LLA | 50 | Crystalline |
| DLLA | 50 | Amorphous |
| LLA | 90 | Crystalline |
| DLLA | 90 | Amorphous |
| LLA | 120 | Crystalline |
| DLLA | 120 | Amorphous |

Polymers having the characteristics described in Table 7 are synthesized as described above, providing direct control over the length of hydrophobic and hydrophilic segments (DP) as well as crystallinity. In addition, direct comparisons with physical properties reported from previous studies on hydrophobically modified PEO can be made by appropriate choice of PEO segment lengths. Detailed evaluation of structure-property relationships and comparison with theoretical models can provide fundamental insight into the nature of hydrogel structure and mechanical properties, leading to novel materials with predictable properties for gels and biological applications.

It is expected that important parameters governing the linear viscoelasticity of physically associated gels are the cross-link density per chain, the interaction strength, and the lifetime of the network junction (Green, M. S., et al. Chem Phys 14: 80-89, 1946); Tanaka, F. and Edwards, S. F. Viscoelastic Properties of Physically Cross-linked Networks: Transient Network Theory. Macromolecules 25: 1516-1523, 1992; Annable, T., et al. The Rheology of Solutions of Associating Polymers: Comparison of Experimental Behavior with Transient Network Theory. J. Theol. 37: 695-726, 1993). More recent models by Tanaka and coworkers have considered directly the interaction strength of the network junction. For example, Tanaka and Edwards describe the shear elastic and loss moduli (G′ and G″) in terms of the chain breakage rate under shear, which is determined by the process of the hydrophobic segment binding to the micellar core. Similarly, Mattice and co-workers define the junction lifetime as a function of the interaction strength (χₐₛ) and report simulations that show the effect χₐₛ has on the elastic modulus. Where a larger χₐₛ gives stronger interactions and increased elastic modulus (Misra-Nguyen, M., et al. Bridging by End-Adsorbed Triblock Copolymers. Macromolecules 29: 1407-1415, 1996). This parameter can be systematically tuned by varying the length of the hydrophobic block, and can be estimated via SANS and other techniques. Finally, work on model ABA copolymer gels by Kornfield and co-workers suggests that the detailed nature of the hydrophobic interactions and self-assembly must also be taken into account. Physically, these can be quantified by the micelle aggregation number, micelle size, and size of hydrophobic domains, all of which can be measured via scattering.

Polymers described above have a constant PEO block length with a MW of 8,900. It is believed that the MW of hydrophilic segments DP_{PEO}/DP_{PLA} ratio, PEO MW, can determine hydrogel properties. For example, water swelling properties can be dependent on PEO block length or DP_{PEO}/DP_{PLA} ratio. In general, the majority of hydrogel studies with PLA-PEO-PLA polymers have focused on very small PEO MW materials, typically less than 7,000 daltons. In addition, most polymer MWs have also been small. Thus, the impact of total MW or PEO MW is not known in these materials although a comparison of different literature reports suggests MW will affect sol-gel compositions and temperature. As a result, it is expected that total MW will impact mechanical properties of the hydrogel in addition to hydrophobe length.

To address the effect of PEO MW, six new polymer samples are prepared with a common DP_{PLLA} of 60. The PLLA block length of 60 is chosen from the preliminary data section because this length produced strong gels with intermediate properties of the three materials that were studied. Therefore, as the PEO length is decreased compared to polymer C2-2, the elastic modulus of the gel is expected to increase as the hydrophobic block has a greater impact. Conversely, longer PEO segments are likely to decrease the elastic modulus as the density of micelles in the gel is lowered. The PEO segments are summarized in Table 8 and include MWs from 1,000 to 32,000. Since the entanglement MW of PEO is ˜1,600, samples D1 and D2 may show a large transition. However, the entanglement MW is most likely to be influenced toward higher MW by the two PLLA end blocks and the transition may be observed between samples D2 and D3. These samples differ in both the PEO MW and DP_{PEO}/DP_{PLA} ratio.

TABLE 8
Copolymers with Various PEO MWs with constant PLLA lengths.

| Sample | MW_{PEO} | MW_{PLLA} | DP_{PEO}/DP_{PLA} |
|---|---|---|---|
| D1 | 1,000 | 4,320 | 0.38 |
| D2 | 2,000 | 4,320 | 0.75 |
| D3 | 4,000 | 4,320 | 1.5 |
| D4 | 16,000 | 4,320 | 6.0 |
| D5 | 24,000 | 4,320 | 9.0 |
| D6 | 32,000 | 4,320 | 12.0 |

Several polymers are prepared as shown in Table 9 to evaluate the roles of total MW or DP_{PEO}/DP_{PLA} ratio in determining gel properties, including strength. The first three polymers are based on findings described above, in which the total MW of polymers is doubled and the DP_{PEO}/DP_{PLA} ratio is held constant. These polymers permit the comparison of the hydrogel properties of polymers that have different MW but identical DP_{PEO}/DP_{PLA} ratio. Additionally, polymers based on PEO lengths described in Table 8 above are prepared with the same DP_{PEO}/DP_{PLA} ratio of 3.3. These designs allow polymer D9 to fill a position in Table 8 between samples D3 and D4.

TABLE 9
Copolymers with Constant DP_{PEO}/DP_{PLA} but with Various MW.

| Sample | MW_{PEO} | DP_{PEO} | MW_{PLLA} | DP_{PLLA} | DP_{PEO}/DP_{PLA} |
|---|---|---|---|---|---|
| D7 | 4,400 | 100 | 2,160 | 30 | 3.3 |
| D8* | 8,900 | 202 | 4,320 | 60 | 3.4 |
| D9 | 13,200 | 300 | 6,480 | 90 | 3.3 |
| D10 | 16,000 | 364 | 7,920 | 110 | 3.3 |
| D11 | 24,000 | 545 | 11,952 | 166 | 3.3 |

These two sets of polymers allow the separation of the effect of PEO block length on hydrogel properties from that of total polymer MW. In addition to the other polymers described in this section, the influence of the PLLA, PEO, and total MW on hydrogel properties can be studied, to develop comprehensive structure-property relationships from these polymers for the first time.

TABLE 10

| | | | | | | Vary Total MW, | |
|---|---|---|---|---|---|---|---|
| Constant PEO, | | | | Vary PEO, | | Constant | |
| Vary PLLA | | PLLA vs. PDLLA | | Constant PLLA | | DP_{PEO}/DP_{PLLA} | |
| MW_{PEO} | DP_{PLLA} | MW_{PEO} | DP_{PLA} | MW_{PEO} | DP_{PLLA} | Total MW | DP_{PEO}/DP_{PLA} |
| 8,900 | 48 | 8,900 | 50 (PLLA) | 1,000 | 60 | 6,560 | 3.3 |
| 8,900 | 60 | 8,900 | 50 (PDLLA) | 4,000 | 60 | 13,220 | 3.4 |
| 8,900 | 72 | 8,900 | 90 (PLLA) | 16,000 | 60 | 19,680 | 3.3 |
| 8,900 | 86 | 8,900 | 90 (PDLLA) | 32,000 | 60 | 23,920 | 3.3 |

The influence of PBS on critical micelle concentration (CMC), micelle size and shape, gel point, micelle aggregation number, elastic and loss modulus, and SPRS can be determined on selected polymers from each group as outlined in Table 10. The ionic strength of PBS is expected to lower the CMC and gel point. The influence on micelle size and aggregation is not known but expected to be minimal. Alternatively, studies can be performed in PBS or salt solutions prepared from deuterated water.

In addition to studying release profiles over time, the structure of the gel can be monitored during the same period, to correlate any changes observed in the release profile with structural changes in the gel. Structural changes in the gel are expected since the PLLA segments are hydrolysable. Also, as described above, most release studies observe release rate changes over time which is most likely due to structural changes in the gel.

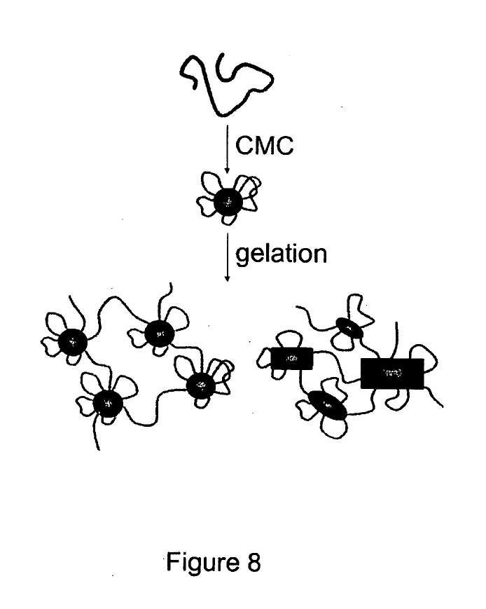

FIG. 8 illustrates two possible gel structures which might be expected from these copolymers. One is formed by interconnected micelles bridged by hydrophilic segments (bottom left) and is generally favored by polymer architectures of the type described here. An alternative gel structure is shown in which the micelles are no longer spherical due to crystallization energies of the hydrophobic domains (bottom right). This type of gel could form when the PLA domains are crystalline and has structural similarities to well known polymer gels formed by swollen polycrystalline materials, like polyethylene. SANS data presented above are consistent with this crystalline gel structure.

DLS and molecular probes (MP) are used to characterize polymer aggregation in the dilute regime. DLS are used to track micelle formation and evolution as concentration and temperature are changed. These two techniques determine CMC and the dependence of these parameters on temperature, presence of buffer or cell media, and added drugs or proteins.

DLS and environmentally sensitive molecular probes can be used as two independent methods to determine CMC. These methods are complimentary and should provide similar CMC values. However, since DLS studies are more difficult, time consuming, and require high purity experimental conditions, correlations between DLS and MP are useful. Once these correlations are developed, MP experiments can be used to quickly obtain the CMC and DLS and used to collect detailed quantitative physical data including micelle size, distribution, and temperature dependence.

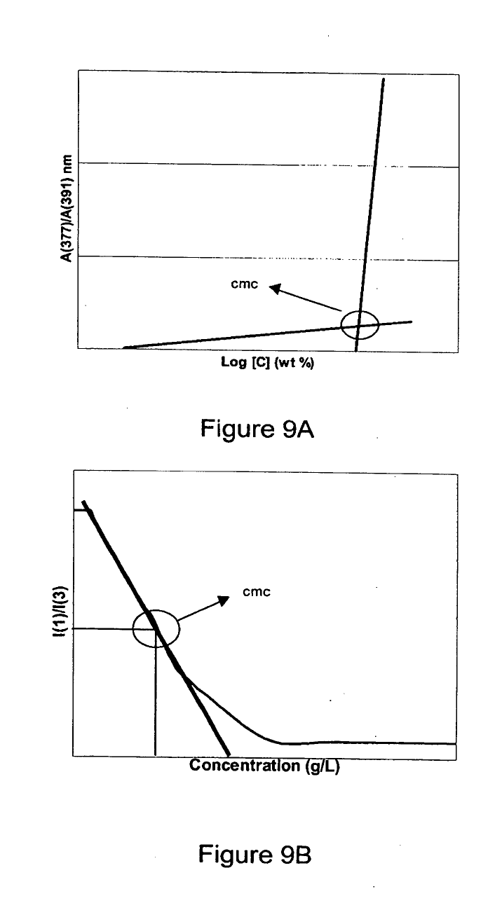

MP experiments are performed using UV-Vis and Fluorescence spectroscopy. For absorption measurements, the hydrophobic dye 1,6 diphenyl-1,3,5-hexatriene (DPH) are used and monitored at 377 and 391 nm. A small aliquot of DPH is added to aqueous polymer solutions of varying concentrations ranging from 0.0005 to 0.5 wt %²⁰ The absorbance ratio (A₃₇₇/A₃₉₁) is plotted versus the log of polymer concentration and two different slopes are determined, as shown in FIG. 9A, when DPH is in an aqueous environment or a hydrophobic environment (micelle formation). The CMC is defined as the intersection of these two lines, and should be expected to be ˜0.005 wt %. Pyrene is used for fluorescence experiments. Similar plots can be prepared but, in this case, the transition is less sharp and CMC is defined as the mid-point of the transition (FIG. 9B) (Liu, L., et al. Micellar Formation in Aqueous Milieu from Biodegradable Triblock Copolymer Polylactide/Poly(Ethylene Glycol)/Polylactide. Polym J 31: 845-850, 1999).

DLS can be used to measure the hydrodynamic radius as a function of polymer concentration, which can be used to determine the onset of aggregation (the CMC). DLS measurements have yielded CMC's in the range of 0.005-0.001 wt %, confirming results from MP experiments. DLS measurements can be easily performed under different conditions to examine the effect of temperature on the micellization process and micelle size. The self-assembly of PLA-PEO-PLA copolymers is shown in FIGS. 1A-1B and 2A-2B to be temperature sensitive. DLS can also provide data on CMC and micelle size under physiologically relevant pH and ionic strength. Further, DLS can be used to determine if proteins or hydrophobic drugs affect the CMC or micelle size, and thus delivery profiles of these molecules from PLA-PEO-PLA gels.

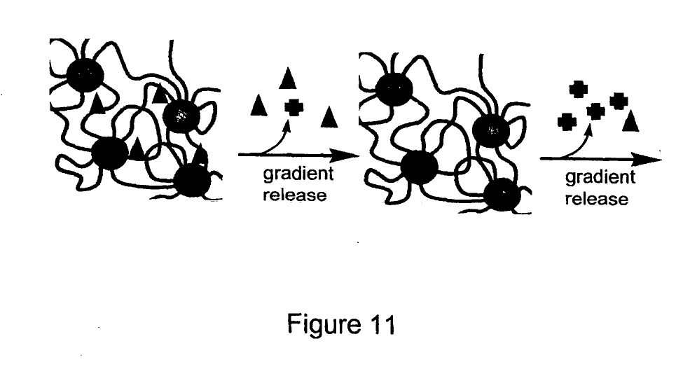

It is believed that the nature of the drug will impact release behavior, with hydrophobic drugs located inside the micelle core while hydrophilic compounds would be found preferentially in PEO domains. When compounds are located in PEO domains or the aqueous continuous phase, these are more likely to experience simple diffusion interactions leading to release from the hydrogel. In contrast, segregation of compounds into hydrophobic micelles would confine theft movement so that release is dependent on micelle structure, reorganization, and degradation. Here, the release profile is expected to follow a reservoir-like model. FIG. 11 illustrates this concept, which is also supported by data presented above. SANS and DLS experiments are used to study the location of the agents relative to gel structure. DLS experiments yield a rapid estimate of whether or not bioactive compounds are increasing the micelle size and swelling the hydrophobic micelle core, which is expected to correlate with the hydrophobicity and micelle-water partition coefficient of the drug compound. If these effects are significant, SANS spectra can be taken in the presence and absence of deuterated bioactive molecules. By utilizing contrast-matching techniques, scattering from the PLA domains, the PEO domains, and the drug can be distinguished. Fitting these spectra with standard models for polymeric micelles can yield insight into whether the bioactive compounds are localized to the micelle core, distributed between the core and the aqueous phase, localized to the micelle interface, and so on. RS can also prove useful for determining if drugs are present in the PLA micelles. These results will then be compared to release profiles to determine whether or not solute location within the gel microstructure impacts release behavior.

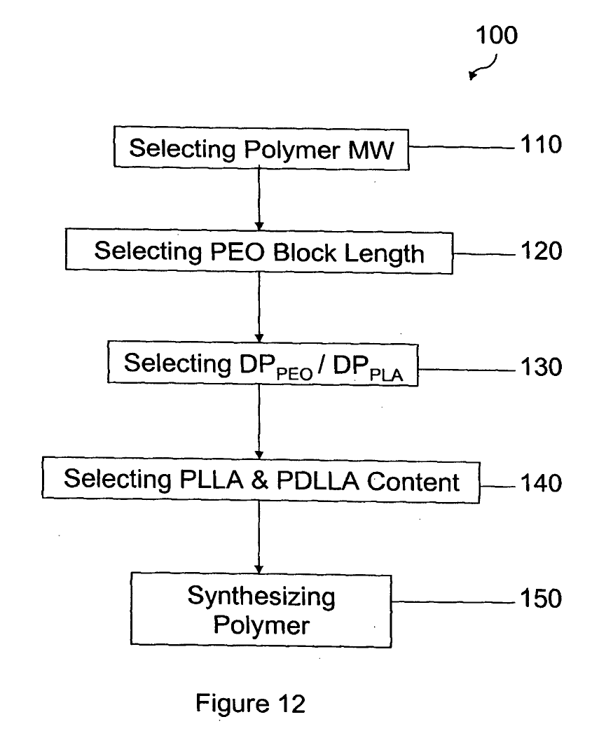

FIG. 12 illustrates an embodiment of a method of designing and synthesizing triblock copolymers. In certain embodiments, the triblock copolymers are PLA-PEO-PLA block copolymers. FIG. 12 is a schematic diagram of an embodiment of a method 100 of designing a triblock copolymer having desired properties, comprising selection of a polymer molecular weight 110; selecting a poly(ethylene oxide) block length 120; selecting a ratio of the degree of polymerization of poly(ethylene oxide) to the degree of polymerization of poly(lactic acid) 130; and selecting a relative content of poly(L-lactic acid) and poly(D,L-lactic acid) 140. The designed triblock copolymer can then be synthesized as described elsewhere herein.

In certain embodiments, parameters of the triblock copolymers are selected to produce hydrogels having desired properties such as elastic modulus or drug release characteristics. Such parameters include MW_{PEO}, DP_{PLLA}, DP_{PLA}, total MW, and DP_{PEO}/DP_{PLLA}. Exemplary embodiments having specific values for such parameters are summarized in Table 11, below.

It should be noted that the sequence of method steps can be varied, but can be chosen by a particular design considerations, e.g., in order to obtain a hydrogel with a desired high elastic modulus, the relative content of poly(L-lactic acid) and poly(D,L-lactic acid) may be selected as all poly(L-lactic acid) before the total molecular weight is selected.

TABLE 11
Summary of triblock copolymer parameters modifiable for control of hydrogel
properties.

| Constant PEO, | | | | Vary PEO, | | Vary Total MW, | |
|---|---|---|---|---|---|---|---|
| Vary PLLA | | PLLA vs. PDLLA | | Constant PLLA | | Constant DP_{PEO}/DP_{PLLA} | |
| MW_{PEO} | DP_{PLLA} | MW_{PEO} | DP_{PLA} | MW_{PEO} | DP_{PLLA} | Total MW | DP_{PEO}/DP_{P} |
| 8,900 | 48 | 8,900 | 50 (PLLA) | 1,000 | 60 | 6,560 | 3.3 |
| 8,900 | 60 | 8,900 | 50 (PDLLA) | 4,000 | 60 | 13,220 | 3.4 |
| 8,900 | 72 | 8,900 | 90 (PLLA) | 16,000 | 60 | 19,680 | 3.3 |
| 8,900 | 86 | 8,900 | 90 (PDLLA) | 32,000 | 60 | 23,920 | 3.3 |
| 8,900 | 120 | | | | | | |
| 8,900 | 200 | | | | | | |
| 8,900 | 400 | | | | | | |
| 8,900 | 800 | | | | | | |

In certain embodiments, selecting a polymer molecular weight can include one or more of a) evaluating overall PLLA and PEO weight percent, b) determining the molecular weight between cross-links formed by the hydrophobic A-blocks, c) determining the degradation rate, d) determining the drug release rates, and e) selecting the overall elastic modulus.

In certain embodiments, selecting a poly(ethylene oxide) block length can include one or more of a) selecting the desirable molecular weight between cross-links, b) selecting the network porosity, c) selecting the PLLA weight percent, and d) selecting the total weight percent polymer of the gel.

In certain embodiments, selecting a ratio of the degree of polymerization of poly(ethylene oxide) to the degree of polymerization of poly(lactic acid) can include one or more of a) determining the weight percent of each polymer block for the desired elastic modulus, b) selecting the desirable molecular weight between cross-links, c) selecting a desired degradation rate, d) selecting a desired drug release rate, e) selecting a desired hydrophobic network domain size, and f) selecting a desired percent of crystallinity.

In certain embodiments, selecting a relative content of poly(L-lactic acid) or poly(D,L-lactic acid) can include one or more of a) evaluating the weight percent of each block, b) selecting a desired degradation rate, c) selecting a desired drug release rate, d) selecting a desired hydrophobic network domain size, and e) selecting a desired percent of crystallinity.

In certain other embodiments, synthesizing the triblock copolymer may include a variety of other methods known in the art, including but not limited to ring-opening polymerization of cyclic lactones and use of a catalyst selected from Zn, CaH₂, SnO, SnO₂, SnCl₂, GeO₂, Al (OiPr)₃, Yt (alkoxides)₃, Na, potassium t-butoxide, Sn (triflate)₂, a N-heterocyclic carbene and a single site nickel catalyst.

The claims should not be read as limited to the described order or elements unless stated to that effect. Therefore, all embodiments that come within the scope and spirit of the following claims and equivalents thereto are claimed as the invention.

### EXAMPLES OF THE INVENTION

The following non-limiting examples and data illustrate various aspects and features relating to the polymeric compounds, compositions and/or methods of the present invention, including the assembly of gel materials and their use in the delivery and release of various therapeutic agents. In comparison with the prior art, the present compounds, compositions and/or methods provide results and data which are surprising, unexpected and contrary thereto. While the utility of this invention is illustrated through the use of several ABA triblock copolymer compounds and related A and B block components used therewith, it will be understood by those skilled in the art that comparable results are obtainable with various other hydrophobic or hydrophilic block components and the corresponding ABA triblock copolymers, as are commensurate with the scope of this invention. For instance, it will be understood by those skilled in the art that comparable results can be obtained by incorporation of one or more polyesters or monomers, of the type described herein, (e.g., poly(3-hydroxybutyrate), poly(4-hydroxybutyrate), poly(3-hydroxyvalerate), poly(caprolactone), poly(valerolactone)) into an A block component. Likewise, various other poly(alkene oxides) or monomers thereof can be incorporated into the B block of one or more of the present triblock copolymeric compounds.

Either L-lactide ((3S)-cis-3,6-dimethyl-1,4-dioxane-2,5-dione)) or racemic lactide (3,6-dimethyl-1,4-dioxane-2,5-dione) from Aldrich was purified by recrystallization in ethyl acetate and then sublimated prior to polymerization. α,ω Dihydroxy polyethylene glycol macroinitiator with molecular weight 8000 (PEG 8K, Aldrich) was dried at room temperature under vacuum for two days prior to polymerization. MALDI and GPC showed this polymer to be 8,900 in weight. Stannous octanoate (Alfa Aesar) was used without further purification.

### Example 1a

General Method for PLA-PEO-PLA triblock reaction. PEO was dried under vacuum for a day prior to use. Both L-lactide and DL-lactide were recrystallized in ethyl acetate, and sublimated prior to use. Tin octonoate catalyst was used without further purification. Telechelic PEO macroinitiator (10.0 g, 1.14 mmol, 1 equiv) was weighed into a dry round bottom flask with a stir bar. The PEO was stirred and heated at 150° C. while purged with nitrogen. Tin octonoate (185 μL, 0.57 mmol, 0.5 equiv) was added to the mixture, followed by immediate addition of L-lactide (6.9 g, 47.9 mmol, 42 equiv). The reaction was capped and reacted at 150° C. for 24 hours. After cooling the reaction was quenched with methanol, diluted with tetrahydrofuran, and precipitated in hexanes. Dissolution and precipitation was repeated 3 more times. The recovered white powder was then dried under vacuum. ¹H NMR spectra were recorded with a 300 MHz Bruker Spectrospin 300. Chemical shifts are expressed in parts per million using deuterated chloroform solvent protons as the standard. Gel permeation chromatography was performed with a Polymer Laboratories PL-GPC50 with 2 PLGel 5 μm Mixed-D columns, a 5 μm guard column, and a Knauer RI detector. The eluent was N,N-dimethyl formamide with 0.05M LiCl at 50° C. ¹H NMR (300 MHz, CDCl₃) δ 5.16, δ3.62, δ1.59, Mₙ=13,958; GPC (DMF) PDI=1.2.

### Example 1b

General method for Rheological data. In a typical method of preparation, nanopure water was added to a measured amount of polymer to make 25 wt % samples. These samples were kept at equilibrium for 1 day at room temperature and then heated at 40° C. for 20 hours. The gels were again allowed to sit for 2 days before being transferred to a TA instruments AR2000 stress controlled rheometer for oscillatory measurements. Rheological measurements were performed using the cone and plate geometry (40 mm diameter cone with a 2° cone angle). A solvent trap was used and water evaporation was not significant for the temperature and timescales investigated. Stress sweep at a constant frequency of 1 Hz was first performed to obtain the linear viscoelastic region for collecting subsequent data. Frequency sweep tests over a frequency range 0.01 Hz to 100 Hz, were done at constant stress amplitudes to measure G′ and G″ (storage and loss moduli, respectively) corresponding to the linear response. The data in FIGS. 4D-E show that the stereoregular sample forms a gel (G′>G″) while the stereorandom sample does not. Using the common definition from the literature, when G′>>G″ then the sample is considered a gel. This again indicates that the stereocenter plays an important role in gel formation. FIG. 4F shows another example in which the stereoregular polymer makes a stiffer gel than the stereorandom polymer. This is true even though the racemic polymer is two units longer than the stereoregular one. These sets of data show that the hydrogels' stiffness is increased when using stereoregular PLA and this is consistent even when the molecular weights are varied. All solutions were prepared as described above except for the 58L and 60R samples. These are 25 wt % gels formed at 80° C.

### Example 1c

General Method for WAXD Data. Wide angle X-ray diffraction measurements of the gels from FIGS. 4G-H (66R and 70L at 20 wt % and formed at 40° C.) were performed on a Panalytical X'Pert Powder Diffractometer from 2θ=5° to 60°. FIG. 4H shows a spectrum for two other triblock copolymer samples, 77L and 78R. The sample formed from stereorandom polymer shows no crystalline peaks while the sample formed from the stereregular polymer shows peaks at 2θ≈17° and 19° indicating crystalline PLLA.

### Example 2

The polymer used in the SANS study are listed in Table 12 were used in the SANS study to assess changes in solution and gel structure, by varying two parameters, the MW and crystallinity of the hydrophobic PLA block. The polymers made from crystalline L-lactic acid block have been named L-lactide series polymers and those made from a amorphous mixture of D/L lactic acid polymers are called R-lactide series polymers. Within each series the PLA block lengths of polymers has been suitably chosen to match the polymer MW in the other series for easy comparison. The sample names indicate the total length of PLA block followed by a letter indicating the stereo specificity of PLA block. Hence 58R refers to the polymer PLA₂₉PEO₂₀₂PLA₂₉ in R-lactide series.

In a typical method of SANS sample preparation, the required concentration of solution of the polymer in D₂O was made which was then stirred for a day at room temperature. The samples were then heated at 80° C. for 20 hours. The polymer solution was again stirred for 1-2 days at room temperature to allow it to reach equilibrium. For each polymer studied, solutions were made at concentrations of 0.5%, 2%, 10%, 22% and 30% by weight for easy comparison.

Small angle neutron scattering (SANS) measurements were conducted on the Small Angle Scattering Instrument (SASI) at the Intense Pulsed Neutron Source located at Argonne National Laboratory, Argonne, Ill. Scattering length densities used for calculations are in Table 13, where ρ_{b}=ρb/MN_{AV}, with N_{AV} being Avagadro's number, b the scattering length density, ρ the bulk density of the polymer, and M the monomer molecular weight. Spectra were obtained at 25° C. for all the samples. Quartz sample cells with a path length of 1 mm and 2 mm were used for the concentrated and dilute samples, respectively. Spectra were collected for one to four hours, depending on the sample concentration and contrast. Deuterated water was used to quantify the solvent scattering, which was subsequently subtracted off from the spectra. Incoherent scattering background was estimated from the signal at high q and was also subtracted from the data. The q-range covered in these experiments was 0.005 Å⁻¹<q<1.0 Å⁻¹. The sample to detector distance was 1.44 m.

TABLE 12

| | PLA | PEO | D₂O |
|---|---|---|---|
| SLD (ρ_{b}) (Å⁻²) | 1.75e−6 | 6.38e−7 | 6.36e−6 |

TABLE 13
PLA-PEO-PLA Triblocks

| Sample | | | | Crystallinity |
|---|---|---|---|---|
| Name | DP_{PLA} | DP_{PEO} | MW | of PLA block |
| 58L | 58(PLLA) | 202 | 13064 | crystalline |
| 72L | 72(PLLA) | 202 | 14072 | crystalline |
| 77L | 77(PLLA) | 202 | 14444 | crystalline |
| 88L | 88(PLLA) | 202 | 15224 | crystalline |
| 60R | 60(PDLA) | 202 | 13208 | amorphous |
| 72R | 72(PDLA) | 202 | 14072 | amorphous |
| 88R | 88(PDLA) | 202 | 15224 | amorphous |
| 92R | 92(PDLA) | 202 | 15524 | amorphous |

### Example 3a

R-Lactide Series Polymers

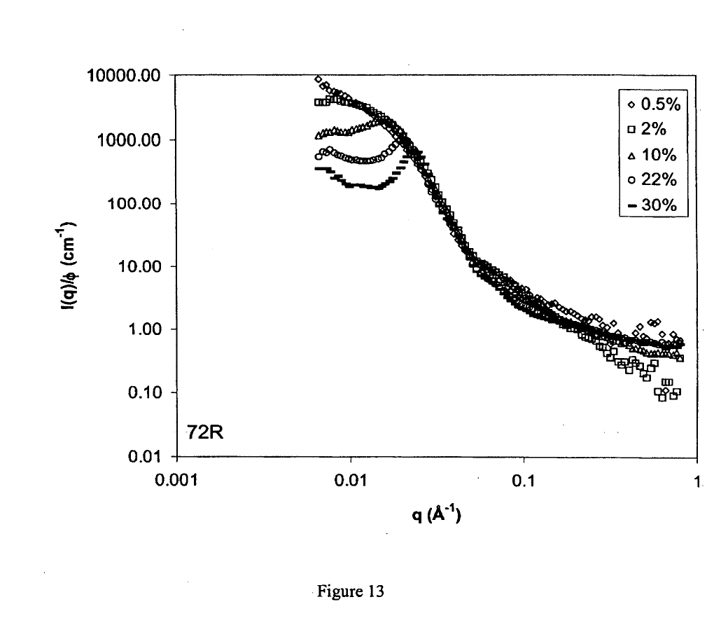

SANS experiments were carried out on the R-series polymers at concentrations of 0.5%, 2%, 10%, 22% and 30% by weight to compare the effect of micelle formation and packing. The scattering spectra for 72R at all the concentrations are shown in FIG. 13 after scaling them with their respective volume fraction. At the lowest concentration of 0.5 wt %, no correlation peak is seen in the scattering spectrum. The spectrum very closely resembles that of spherical micelles in solution. Hence at the lowest concentrations individual micelles are expected to be present in solution. As the concentration of polymer is increased in solution the micelles come closer to each other and tend to associate with neighboring micelles through the hydrophobic ends to form a network of associated micelles. This leads to ordering of the cores of the micelles, which shows up in the form of a correlation peak for concentrations above 10 wt %. This peak becomes sharper and shifts to higher wave vectors, q, with increasing concentrations (FIG. 13), indicating improved long range order and a decrease in lattice spacing as the micelles get more packed and bridges between them increase in number.

### Example 3b

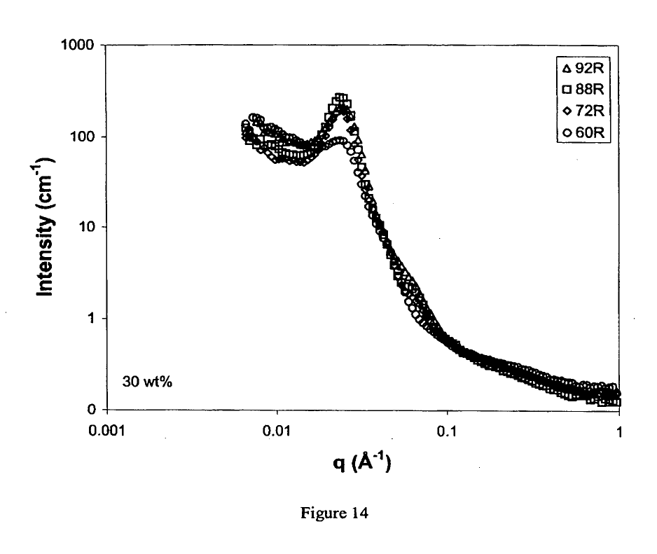

At fixed concentration, as the block length of PLA is changed, no change in the correlation distance between the micelles is observed (FIG. 14). Also the scattering spectra overlaps for all the polymers in the low and mid q range irrespective of the length of the PLA block showing that the internal structure of the micelles remains unchanged for all polymers of the R lactide series. There is a noticeable trend though, in the slope of the scattering spectra in the high q range. The slope is seen to increase (become less negative) as the PLA block length is decreased. The scattering at large q values is obtained because of monomer-monomer correlations arising out of ‘blob’ scattering in the micelle corona. The scattering intensity at large q goes as q^{-I/v} where ν is the Flory exponent and is ⅗ for swollen polymer chains in a good solvent (chains with excluded volume interactions). (Auvray, L.; De Gennes, P. G. Europhys. Lett. 1986, 2, 647-650. Cosgrove, T.; Hone, J. H. E.; Howe, A. M.; Heenan, R. K. A. Langmuir 1998, 14, 5376-5382.) The value of ν that we obtained for different polymers studied is listed in table 14 and table 15. The agreement with the expected value of ⅗ is clearly seen at high concentrations. These values were calculated after subtraction of the incoherent scattering backgrounds from raw data.

TABLE 14

| | polymer concentration (wt %) | | | | |
|---|---|---|---|---|---|
| | 0.5 | 2 | 10 | 22 | 30 |
| Flory Exponent (ν) for 72R | 0.5 | 0.46 | 0.52 | 0.58 | 0.61 |
| Flory Exponent (ν) for 72L | 0.54 | 0.54 | 0.59 | 0.6 | 0.62 |

TABLE 15
R - Lactide Series

| | PLA block length | | |
|---|---|---|---|
| | 72 | 88 | 92 |
| Flory Exponent (ν) | 0.61 | 0.6 | 0.58 |

L-Lactide Series

PLA block length

| | 58 | 72 | 77 | 87 |
|---|---|---|---|---|
| Flory Exponent (ν) | 0.63 | 0.62 | 0.61 | 0.6 |

### Example 3c

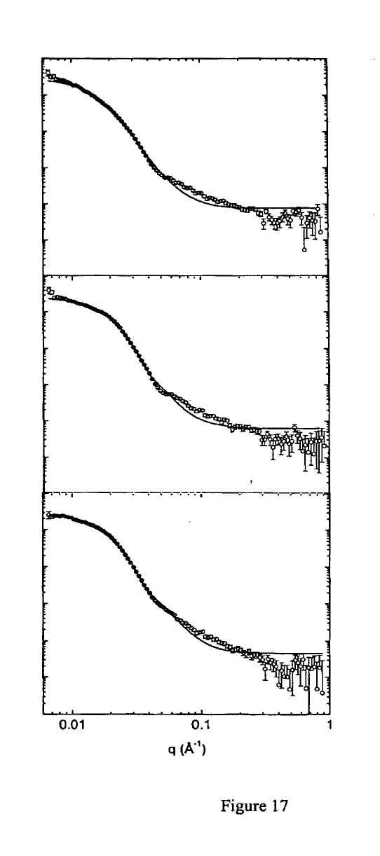

The value of p is representative of the conformation of the polymer in solution and increases from ⅓ (coil like globules) to 1 (rodlike) as the polymer chains are more stretched. (Higgins J. S.; Benoit H. C. Polymers and Neutron Scattering; Claredon Press: Oxford, 1994.) For gaussian chain conformation, the value is ½. As the PLA chain length is increased, the value of ν is seen to increase slightly which indicates that the PEO chains in the corona are more stretched for shorter PLA blocks. This may be explained by the fact that for a smaller length of PLA, the average distance between two micelle centers is less in solution, which increases the possibility of bridging between them (reference to reduction in free energy). Since the chains forming bridges are more stretched than those forming loops or dangling ends, the value of ν shows more bridges being formed in polymer gels with shorter PLA blocks. Also in FIG. 13 the slope at high q is seen to increase (become less negative) and correspondingly value of p is seen to increase from ½ (gaussian chains) to ⅗ (table 14), as the concentration of the polymer in solution is increased. This again shows the fact that as the micelles are packed closer to each other with increasing concentration of polymer, bridging between them increases. This eventually leads to formation of a well-connected phantom network of micelles (FIG. 17), thereby leading to formation of a strong gel. The position of the correlation peak hence indicates the average distance between two micelle centers in the solution. For the 30 wt % polymer solution, this correlation distance is found to be 2π/qₘₐₓ=25.26 nm. The end-to-end distance of PEO chain in unperturbed state is calculated as (r₀²)^{1/2}=(c_{∞}nl²)^{1/2}, where c_{∞}, the characteristic ratio for PEO is 14.44 and the monomer length l is taken to be 3.5 Å. (Kenworthy, A. K.; Hristova, K.; Needham, D.; McIntosh, T. J.; Range and magnitude of steric pressure between bilayers containing phospholipids with covalently attached Poly(Ethyleneglycol), Biophysical Journal, 1995a, 68, 1921-1936. Tonerli, A. E.; Flory, P. J. Macromolecules 1969, 2, 225.) This gives <rₒ²>^{1/2} as 18.9 nm for PEO block length of 202. Similarly the end-to-end distance for PLA block in unperturbed state is calculated as 3.1-3.5 nm for each PLA end block in the various polymers used (c_{∞}=2,l=3.7 Å). (Tonerli, A. E.; Flory, P. J. Macromolecules 1969, 2, 225.) This yields the total end-to-end distance for a chain bridging between two micelles as 25.1-25.9 nm, which is in excellent comparison with the correlation length obtained for the gel network.

### Example 4a

### L-Lactide Series Polymers

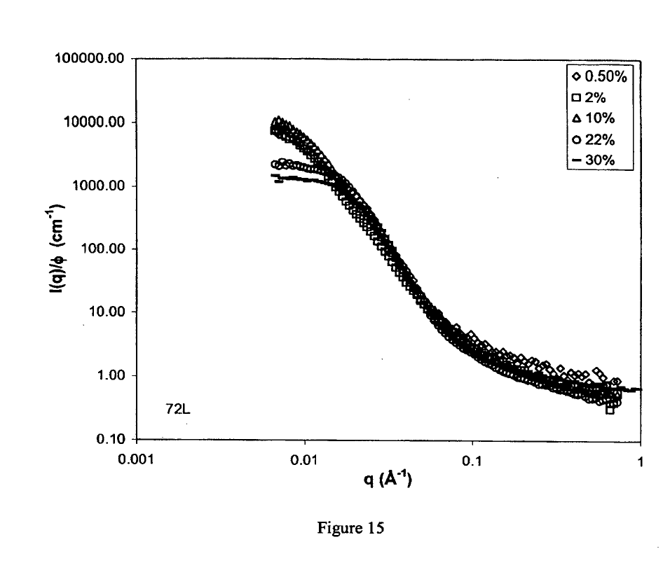

For the polymers made from L-lactide blocks, the scattering spectra obtained was significantly different from that obtained for polymers made from amorphous D-L lactide blocks (FIG. 15). Specifically, no correlation peak was seen to form as the concentration of polymer was increased in this system, though formation of a shoulder was clearly evidenced in the low q regime (see arrow drawn in FIG. 15). Also in the mid and high q region the scattering spectra is seen to overlap completely, similar to what was observed for the R-lactide series polymers, which shows that the internal structure of the aggregates or the form factor of the aggregates in solution does not change as the concentration is increased. The shoulder can be viewed as a very broad correlation peak, which forms as structure factors set in. The formation of the shoulder represents a broad polydispersity in the inter-aggregate spacing in the case of L-lactide series polymers. This polydispersity in correlation length is attributed to crystallinity of the L-lactide block in solution. It has been confirmed by WAXS studies that PLLA domains in solution are crystalline, because of which the L-lactide series of polymers do not form spherical micelles. The PEO chains are expected to align as brushes on the back of PLLA lamellas as has been observed by researchers for other semicrystalline polymers. These chains associate with neighbouring lamellas to form a phantom network structure at high concentrations similar to that formed in R-lactide series polymers. Because of random orientation of the lamellas and polydispersity in their sizes, no well-defined correlation length exists in these systems and hence no peak is observed in their SANS spectra.

### Example 4b

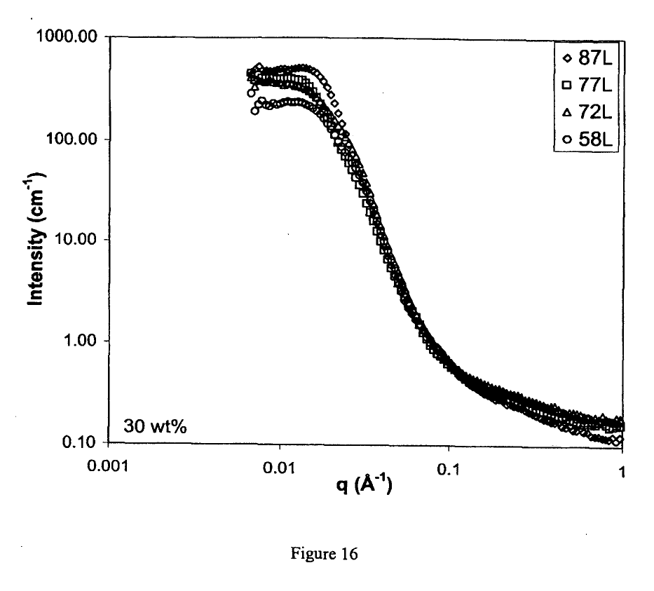

The SANS spectra of various polymers in the L-lactide series overlaps on top of each other (FIG. 16), showing that the nature of the cross linked aggregates formed remains unchanged on changing the length of the PLLA block. Also the slope of the scattering spectra in the high q range is seen to increase as the PLLA block length is decreased, as was seen in the case of R-lactide series polymers, indicating that lower molecular weight polymer chains are more stretched (see ν values, Tables 14 and 15).

### Example 5

Aggregation Number of Hydrophobic Core

The aggregation number of the hydrophobic core of the micelles can be estimated by using the extrapolated absolute scattering intensities as q→0 for dilute solutions of polymers. In dilute regime (0.5 wt %), I(q→0,φ) can be expressed as n(φ)(Δρ)²ν², where n(φ) is the number density of the scattering object (micelles), Δρ is the excess scattering length density of the polymer with respect to D₂O and ν is the volume of the aggregate in bulk. Using this relation the aggregation numbers were obtained for various polymers using extrapolated I(q→0) data. The values of aggregation numbers obtained are listed in Table 16.

TABLE 16
R - Lactide Series

| | PLA blocks length | | |
|---|---|---|---|
| | 72 | 88 | 92 |
| Aggregation Number (N_{agg}) | 114 ± 1 | 102 ± 1 | 97 ± 1 |

L-Lactide Series

| | PLA block length | | |
|---|---|---|---|
| | 58 | 72 | 77 |
| Aggregation Number (N_{agg}) | 191 ± 4 | 198 ± 4 | 165 ± 3 |

### Example 6a

Model Fits to SANS Data

R-Lactide Series Polymers

The simplest form factor model that closely describes spherical anphiphilic micelle formation is the core-corona model. This model accounts for a spherical core of radius R₁ surrounded by a spherical shell of radius R₂. The model also accounts for the difference in contrast between the core and shell and between the shell and surrounding medium. In our study the core is formed by PLA, shell is formed by PEO and D₂O forms the surrounding medium. The overall scattering intensity of the micelle can be written as

$$I ( q ) = {N ( Δρ )}^{2} P ( q ) S ( q ) \\ {( Δρ )}^{2} P ( q ) = {[ \frac{4}{3} {πR}_{1}^{3} ( {ρ}_{1} - {ρ}_{2} ) \frac{3 {j}_{1} ( {qR}_{1} )}{{qR}_{1}} + \frac{4}{3} π {R}_{2}^{3} ( {ρ}_{2} - {ρ}_{s} ) \frac{3 {j}_{1} ( {qR}_{2} )}{{qR}_{2}} ]}^{2} \\ {j}_{1} ( x ) = \frac{sin x - x cos x}{{x}^{2}} \qquad ( 1 )$$

where I(q) is the measured scattered intensity, N is the number density of scattering centers (micelles), (Δρ)²P(q) is the combined contrast and intraparticle form factor, S(q) is the interparticle structure factor, and ρ₁, ρ₂, and ρ_{S} are the scattering length densities of the core, corona and solvent, respectively. (Chen, S. H. Annu. Rev. Phys. Chem. 1986, 37, 351.) Polydispersity in the size of micelles is introduced in this model by averaging the form factor over a Shulz distribution of radii. The presence of water in the core and corona is also included into the model by introducing corrections for the scattering length densities ρ₁ and ρ₂. Hence if α₁ and α₂ are the volume fractions of PLA and PEO in the core and corona respectively, then effective SLD of core and corona is given by,
ρ_{1eff}=α₁ρ₁+(1−α₁)ρ_{S}
ρ_{2eff}=α₂ρ₂+(1−α₂)ρ_{S} (2)
(Goldmints, I.; von Gottberg, F. K.; Smith, K. A.; Hatton, T. A. Langmuir 1997, 13, 3659-3664.) The volume fractions α₁ and α₂ are not independent parameters but can be expressed in terms of three independent parameters R₁, R₂ and micelle aggregation number (N_{agg}) as

$${a}_{1} = \frac{3 {N}_{agg} {V}_{PLA}}{4 π {R}_{1}^{3}} \\ {a}_{2} = \frac{3 {N}_{agg} {V}_{PEO}}{4 π ( {R}_{2}^{3} - {R}_{1}^{3} )} \qquad ( 3 )$$

V_{PLA} and V_{PEO} are bulk volumes of PLA and PEO homopolymers respectively. This model thus has four fitting parameters, namely, the radius of the micellar core, R₁, the radius of the shell, R₂, the micelle aggregation number (N_{agg}) and the polydispersity (σ) in the micelle size. The ratio of R₁/R₂ has been kept constant while introducing polydispersity in order to reduce the number of independent parameters.

### Example 6b

The model of the preceding example was used to fit data for R-lactide series polymers at the most dilute concentration (0.5 wt %) at which the structure factor S(q) can be assumed to be 1. The representative fits of the model to the data for 72R, 88R and 92R samples are shown in FIG. 17. This model assumes homogeneous scattering length densities for the core and the shell while not taking into account the internal polymer structure and monomer-monomer interactions of the chains in solution because of which this model is expected to break down at values of q higher than the inverse correlation length of the internal structure. For q values smaller than the inverse correlation length of the internal structure the core and shell appear uniform in densities and the model is seen to represent the data extremely well. In the high q range the data can be represented by power law contributions of the form q^{-1/ν} . . . which we have analyzed separately to obtain information the conformation of the polymers chains in solution (Tables 14 & 15). The fit parameters obtained from the model are summarized in table 6. The total size of the micelles (R₂) obtained ranges from 9.1 nm for 72R sample to 10.3 nm for 92R sample. Hence in a highly concentrated, well-packed system, center-to-center distance between any two adjacent micelles interacting via hard sphere repulsions would lie in the range 18.2 (72R) to 20.6 (92R) nm. Comparing with the correlation distance obtained in these systems for the 30 wt % gel samples found earlier, it can be deduced that the micelles are not closely packed at this concentration. The micelles repel each other due to osmotic forces, which is balanced by the attractive forces of the bridges joining them. Looking in table 17, as expected, the general trend is that the core radius increases with an increase in the MW of the PLA block. Owing to the constant length of PEO block used, the shell thickness remains constant for all the three cases. The increase in micelle aggregation number with increasing PLA block length is consistent with what researchers have seen in other amphiphilic triblock systems. The values of N_{agg} obtained by model fitting match well the values obtained using the extrapolated I(q→0) values. The volume fraction of PLA and PEO in the core and shell respectively, calculated using equation set (3), shows that both the core and corona are hydrated. The hydration of the core reduces as the length of the hydrophobic group is increased.

TABLE 17

| | PLA block length | | |
|---|---|---|---|
| | 72R | 88R | 92R |
| Core radius | 59.6 ± 0.7 | 67.95 ± 0.29 | 72.16 ± 0.25 |
| (R₁) (Å) | | | |
| Shell thickness | 31.5 ± 0.24 | 33.00 ± 0.21 | 31.03 ± 0.18 |
| (R₂ − R₁) (Å) | | | |
| Polydispersity | 0.3677 ± 0.0068 | 0.2374 ± 0.0031 | 0.2337 ± 0.0026 |
| (σ) | | | |
| Aggregation | 72 ± 2 | 104 ± 1 | 110 ± 1 |
| number (N_{agg}) | | | |
| PLA volume | 0.5577 | 0.6631 | 0.6118 |
| fraction in core | | | |
| (a₁) | | | |
| PEO volume | 0.4161 | 0.4563 | 0.4769 |
| fraction in | | | |
| corona (a₂) | | | |

### Example 7a

L-Lactide Series Polymers

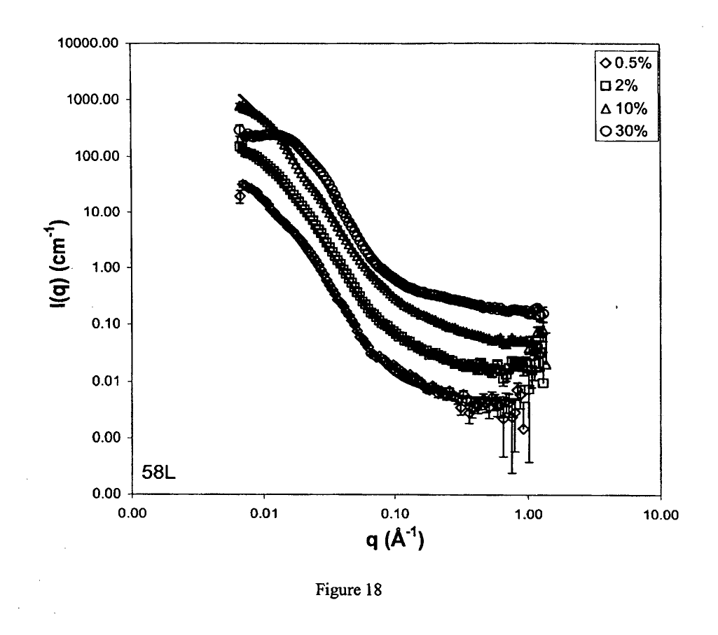

As discussed earlier, the L-lactide polymers are expected to form structures similar to randomly crosslinked, polydispersed network of polymers aggregates. Several groups have previously described scattering from such polydispersed networks. In these systems scattering comes from concentration fluctuations at two length scales; dynamic concentration fluctuations corresponding to thermal fluctuations in semidilute polymer solutions and static concentration fluctuations arising because of long range random inhomogenities or frozen-in constraints in the network such as crystalline zones. Scattering from the two contributions can be combined to give a total structure factor which is a combination of Ornstein-Zernike(O-Z) and Debye-Beuche (D-B) terms, and is expressed as

$$I ( q ) = \frac{{I}_{1} ( 0 )}{1 + {q}^{2} {ξ}^{2}} + \frac{{I}_{2} ( 0 )}{{( 1 + {q}^{2} {ς}^{2} )}^{2}} \qquad ( 4 )$$

where ξ is related to the correlation length of the thermal concentration fluctuations and ξ is representative of the size of the random inhomogenities. The above model was used to fit the scattering spectra for L-lactide series polymers at all concentration ranges, using I₁(0), I₂(0), ξ and ξ as the fit parameters. Incoherent background scattering was accounted for by adding a constant term to the above model. Representative fits for 58L polymer at different concentrations ranging from 0.5 wt % to 30 wt % is shown in FIG. 18. The model shows excellent agreement with data at all concentrations except 30 wt % sample, for which deviations occur in the low q range because of inter-aggregate interactions.

### Example 7b

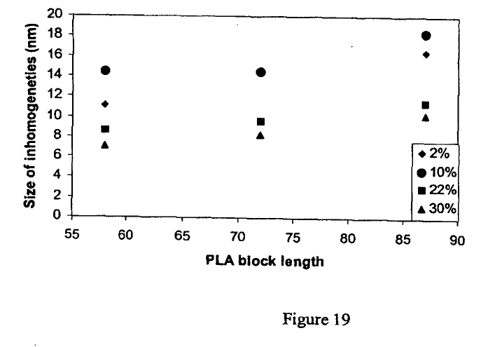

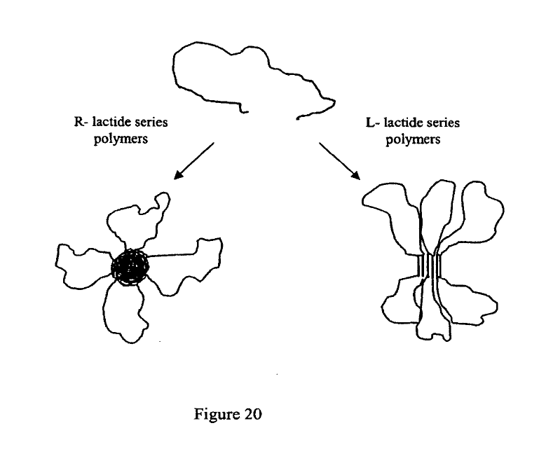

The values of parameter ξ obtained by fitting equation (4) to the scattering data are shown in FIG. 19. Values for parameter ξ was found for all the systems to be very small and unphysical to represent true correlation lengths in the system. This may occur because the density fluctuations due to presence of inhomogenities (ξ) may not be very different in length scale as compared to the correlation lengths (ξ) in the system. The size of inhomogenities is seen to range from 7 to 15 nm and is comparable to the micelle sizes obtained for R-lactide series polymers. Also the sizes of domains are seen to increase with PLA block length similar to the R-lactide series polymers.

### Example 8

With regard to the drug release studies, the copolymers shown in Table 18 were prepared by ring-opening polymerization of L or D-L lactide at 150° C. in the bulk using stannous (II) 2-ethylhexanoate as catalyst. As discussed above, this method is known to limit racemization of the stereocenter and produce polymers of significant molecular weight and narrow polydispersity. The macroinitiator, PEO, has molecular weight (MW) of 8,900 daltons, and four different polymers were prepared with increasing PLA block lengths. These lengths varied from a total DP of 58 to 88 so that the total lactide composition was always smaller than PEO. ¹H-NMR integration was used to establish the M_{w} for PLLA blocks as opposed to GPC standards. In all cases, the polymerization was not run to completion since this broadens the molecular weight distribution.

A comparative study of PLA-PEO-PLA copolymers was performed, where the PLA block was made from L-lactide (LLA), which forms crystalline hydrophobic domains, versus those made from D-L lactide, which forms amorphous domains. Four sets of polymers were made for either series with varying lengths of the PLA block, but constant length of the PEO block. The block lengths of polymers chosen in the two series were equal or close for direct comparison. Table 18 lists the different materials that were used in this study.

TABLE 18
Characteristics of PLA-PEO-PLA triblock copolymers synthesized

| Copolymer | | | | | Total |
|---|---|---|---|---|---|
| Series | Sample | M_{nPEG}^{a} | M_{nPLA}^{b} | M_{Total} | DP_{PLA}^{b} |
| L-lactide | 58L | 8,900 | 4176 | 10,772 | 58 |
| series | 72L | 8,900 | 5184 | 12,644 | 72 |
| | 77L | 8900 | 5544 | 14,444 | 77 |
| | 88L | 8,900 | 6336 | 13,220 | 88 |
| R-lactide | 60R | 8,900 | 4320 | 10,772 | 60 |
| series | 66R | 8,900 | 4752 | 13,652 | 66 |
| | 72R | 8,900 | 5184 | 12,644 | 72 |
| | 88R | 8,900 | 6336 | 13,220 | 88 |

^{a}Determined by MALDI_TOF and GPC;
^{b}Determined by ¹H NMR

In a typical method of sample preparation, a 5 wt % solution of polymer was made in 0.13M pH=7.4 phosphate buffer solution (PBS) which was stirred for 1-2 days. The solution was then heated at 80° C. for 20 hours, and again stirred for 1 day at room temperature to allow it to reach equilibrium. We have shown that at these conditions R-lactide series polymers form spherical micelles, while L-lactide series polymers form polydisperse micellar aggregates. A premeasured mass of drug was added to the polymer solution and the solution was again stirred until no solid drug particles were visible in the solution. For ease of comparison, the same amount of drug was added for all the different polymer samples considered. A polymer solution containing tetracaine was also prepared in nanopure water, using the same method as mentioned above, for comparison with the sample made in PBS.

### Example 10

The drug loaded polymer solutions were enclosed in cellulose ester dialysis membrane bags with a cutoff molecular weight of 1000 g/mol. A membrane with this molecular weight cutoff allows only the drug to pass through, retaining the polymer. These membrane bags containing drug loaded polymer solutions were suspended in either PBS or in nanopure water. The volume of the buffer was kept very large as compared to the volume of the drug solution. The buffer solution was constantly stirred in order to keep the concentration of drug in the buffer uniform. The entire assembly was maintained at a constant temperature of either 37° C. (for solutions made in PBS) or 25° C. (for solutions made in nanopure water) during the course of experiment. One milliliter of the buffer solution was sampled periodically in order to measure the drug concentration. The drug concentration was measured using UV spectroscopy at 229 nm for sulindac and 310 nm for tetracaine, using calibration curves obtained from standard solutions. In order to maintain the same release conditions, the withdrawn aliquot was poured back into its original flask after UV measurements.

### Example 11

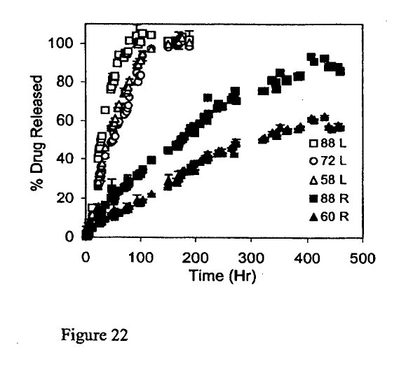

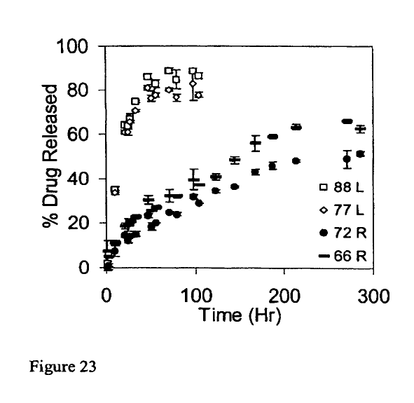

The in-vitro release profiles of sulindac from the different polymers are shown in FIG. 22 and for tetracaine in FIG. 23. Release profiles of tetracaine at 25° C. from polymer solutions prepared in nanopure water are also shown in FIG. 23. The ordinate of the plot is calculated on the basis of cumulative amount of drug release with respect to the amount of drug initially loaded in polymer solution.

Note the unique drug release behavior that is achieved. First, a slow and continuous release of both the hydrophobic drugs, sulindac and tetracaine, was observed to take place over the period of 4-20 days. Secondly, the release rate is seen to be highly dependent on the polymer composition and stereo-specificity of the PLA block. Hence, by simply modifying these variables, the release rate of the drugs can be controlled. This provides a direct and simple way to tune the drug release behavior of these polymer microcarriers.

As shown in FIGS. 22 and 23, the rate of release changes significantly by changing the stereospecificity of the PLA block. Polymers made from L-lactide groups show a much faster rate of release as compared to those made from racemic lactide with the same chemical composition. This trend is consistent with both drugs studied here. The release of sulindac from the L-lactide series of polymers takes place in 4-8 days whereas drug release from the R-lactide series took up to 18 days (FIG. 22). It was also seen that the drug release rate could be modified directly by a small change in the length of the hydrophobic block. The rate of release of sulindac is seen to increase as the PLA block length is increased for both L-lactide and R-lactide series.

### Example 12

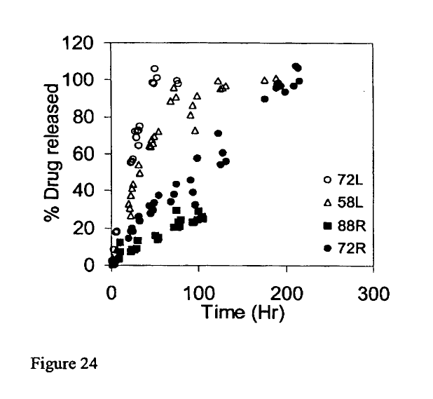

For tetracaine, a slow and nearly zero order release rate is also observed. The release takes place over 2-4 days for L-lactide series, compared to the R-lactide series in which release takes up to 8-9 days (FIG. 23). With an increase in block length of the PLA block, a slower rate of release is observed for the R-lactide series. The rates of release for the L-lactide series appear similar but are again much faster than the R-lactide series. A very similar pattern was observed for tetracaine release in nanopure water at 25° C. (FIG. 24), though in this case the release rate of drug from the R-lactide polymer with a total PLA block length of 72 was seen to be faster than the release rate from polymeric aggregates in PBS. It is also to be noted that the difference in release rates between the two R-lactide series polymers is smaller in the case of tetracaine/PBS system (FIG. 23), than those observed in sulindac/PBS system (FIG. 22), most likely due to the fact that the R-lactide series polymers used have a similar molecular weight in FIG. 23 (difference of 6 PLA units in FIG. IV compared to 28 PLA units in FIG. 22).

### Example 13

In all cases, the systems did not show a burst type release profile showing that there are likely strong interactions between the drug and PLA. These interactions can either be strong hydrophobic binding between PLA and the drug or formation of reversible polymer-drug linkages like hydrogen bonds. Hence, drug release takes place as a result of two factors; overcoming the polymer-drug interactions and diffusion of the drug through the highly crowded polymer matrix.

It is known that the solution of PLA-PEO-PLA in nanopure water is acidic because of degradation of the PLA block. The acidic conditions may modify the interactions between the polymer and drug, thereby affecting the drug release rate. This would explain the observed faster release rate of tetracaine from polymer solutions in nanopure water (FIG. 24) compared to those in PBS (FIG. 23).

### Example 14

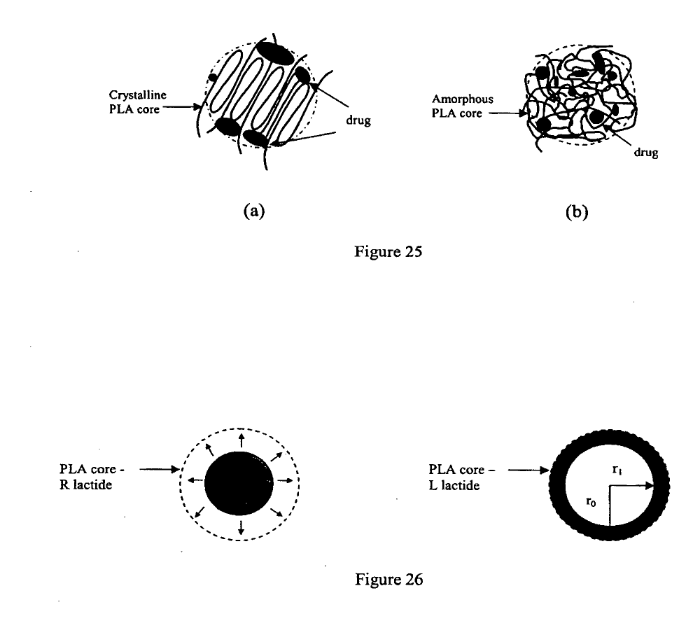

The large difference in release rates observed between L and R lactide series can be attributed to the difference in their crystallinity. L-lactide forms a highly packed crystalline micellar core, preventing the polymer chains from adjusting themselves and allowing the drug to be dispersed homogeneously inside the core. Hence the drugs likely lie in small regions along the periphery of the core, allowing them to be released easily into the surrounding medium (FIG. 25A). R-lactide, on the other hand, is amorphous and allows for the drug to distribute homogeneously inside the core (FIG. 25B). The release of drug from the core in the latter case is more difficult, thereby resulting in much slower release rates. This occurs in both the tetracaine and sulindac data.

### Example 15

The effect of increasing PLA block length is not clear, and may result in two competing effects. Increase in PLA block length may likely result in a stronger polymer-drug interaction, but may also lead to tighter packing in the core. The latter effect seems to be more important for the L-lactide series, where the polymers with longer PLA chains are seen to release faster. For the R-lactide series, the longer PLA block polymer should release slower because of increased hydrophobic interactions with the drug, which is what we see in the case of tetracaine. For sulindac, the release rate increases with increase in PLA block length.

### Example 16

A possible mechanism for release of drugs from the polymer core could include two steps; the drug overcomes an energy barrier corresponding to interaction between the polymer and drug and then diffuses out of the polymer matrix into the solvent medium. A simplified representation of this process is depicted in FIG. 26A.

To obtain a mathematical description of the process, consider a case where overcoming the polymer-drug interaction is a much slower step as compared to the diffusion of drug through the polymer. The shaded region in the figure represents the PLA core holding the drug. Assume that the drug is uniformly distributed in the core of the micelle. Expect this assumption to be fairly accurate for the R-lactide series of polymers but not for the L-lactide series. As the drug at the boundary is released, the boundary of the shaded region moves inwards. Assume that the rate constant for overcoming the polymer-drug interaction (k) is constant and the radius of the core does not change. The rate of breakage of the polymer-drug bond should be proportional to the surface area of the interface between the drug loaded and drug-depleted region in the core. Then the rate of release of drug from the core can be expressed as

$$\frac{{dM}_{t}}{dt} = 4 π {r}^{2} k \qquad ( 5 )$$

where dMₜ is the mass of drug released in infinitesimal time dt and r is the radius of the drug loaded region in the core (the shaded region in the FIG. VII(a)). For uniform distribution of drug within the core, we can express its mass density as

$$ρ = \frac{{M}_{0}}{\frac{4}{3} π {r}_{0}^{3}} \qquad ( 6 )$$

Thus the mass of drug released from an infinitesimally thin shell of radius dr is
dM₁=−4πr²ρdr (7)
Combining equations (5) and (7) and integrating we have

$$\begin{matrix}{\int }_{0}^{t} k ⅆ t = - {\int }_{{r}_{0}}^{r} ρ ⅆ r & ( 8 ) \\ r = {r}_{0} - \frac{k}{ρ} t & ( 9 )\end{matrix}$$

Also integration of equation (7) gives

$${M}_{t} = {M}_{0} - \frac{4}{3} π {r}^{3} ρ \qquad ( 10 )$$

Combining equations (9) and (10) and normalizing with respect to M₀, we get the fractional drug released in time t as

$$\begin{matrix}\frac{{M}_{t}}{{M}_{0}} = 1 - {( 1 - at )}^{3} & ( 11 ) \\ a = \frac{k}{ρ {r}_{0}} & ( 12 )\end{matrix}$$

From the drug release profiles for the L-lactide series, the drug is believed to reside on the periphery of the PLA core. Since the above model assumes uniform distribution of the drug in the core the model can be modified slightly to explain the release profiles in the L-lactide series. In this case, assume uniform distribution of the drug in a thin shell along the periphery with inner radius r₁ and outer radius r₀ (FIG. 26B). Consequently the mass density of the drug becomes

$$ρ = \frac{{M}_{0}}{\frac{4}{3} π ( {r}_{0}^{3} - {r}_{1}^{3} )} \qquad ( 13 )$$

The equation (11) is still valid for the rate of drug release from the L-lactide series with a modified value of ρ.

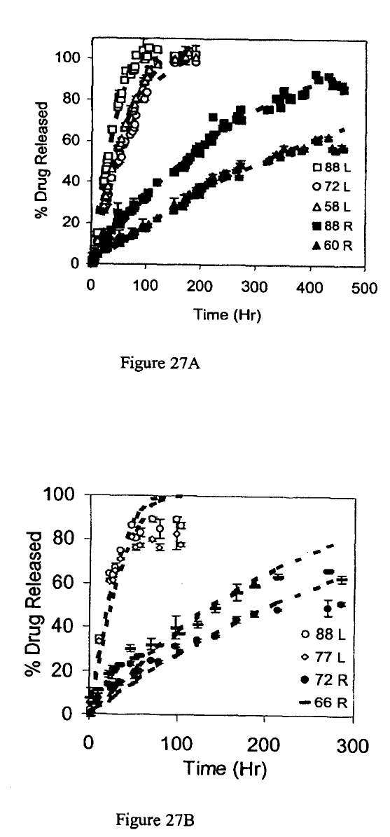

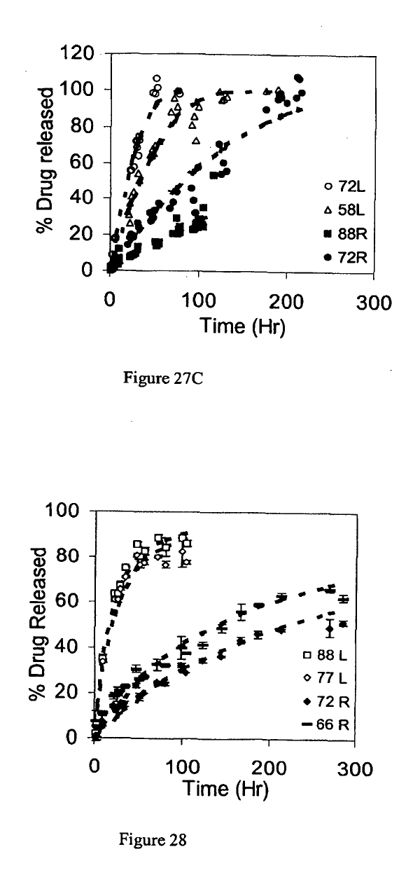

The fit of the release profiles to the equation (11) is done by least square method and the fits are shown in FIG. 27. The model fits the data very accurately for sulindac and reasonably well for tetracaine. The values of the fit parameter α obtained are listed in table 19.

TABLE 19
Parameter α obtained by fitting equation (7) to the release profiles

| Copolymer | | a_{sulindac} | a_{tetracaince/PBS} | a_{tetracaine/water} |
|---|---|---|---|---|
| Series | Sample | (hr⁻¹) | (hr⁻¹) | (hr⁻¹) |
| L-lactide | 58L | 0.00516 | — | 0.00624 |
| series | 72L | 0.00435 | — | 0.01131 |
| | 77L | — | 0.00902 | — |
| | 88L | 0.00798 | 0.0106 | — |
| R-lactide | 60R | 0.00067 | — | — |
| series | 66R | — | 0.00146 | — |
| | 72R | — | 0.00101 | 0.00252 |
| | 88R | 0.00124 | — | 0.00104 |

Since the model fits for tetracaine in PBS at 37° C. were not found optimal, the model was revised slightly by taking a first order dependence of the mass of the unreleased drug on the release rate in equation (5). The final expression for the fractional drug released in time t is obtained as

$$\begin{matrix}\frac{M}{{M}_{0}} = 1 - \frac{1}{{( 1 + {a}^{′} t )}^{\frac{3}{2}}} & ( 14 ) \\ where {a}^{′} = \frac{4}{3} π {kr}_{0}^{2} & ( 15 )\end{matrix}$$

Equation (14) fits the tetracaine in PBS data extremely well (FIG. 28), indicating a different mechanism for breaking of the polymer-drug linkage in this case.

The rate of release as predicted by equation (11) is

$$\frac{1}{{M}_{0}} \frac{ⅆ M}{ⅆ t} = 3 {a ( 1 - at )}^{2} \qquad ( 16 )$$

Hence when at <<1, the rate of release approaches zero order. The release profiles of tetracine and sulindac are seen to be very close to zero order even for very long times.

As shown above, the present invention affords novel drug release behavior of the anti-inflammatory drug sulindac and the local anesthetic tetracaine from aggregates of PLA-PEO-PLA triblock copolymers in solution. Zero order release rates extended over a period of 4-20 days were achieved for these drugs. The release rates of the drugs were shown to be directly dependent on the crystallinity and the molecular weight of the hydrophobic PLA block. The release rate was seen to be much faster from polymers made with crystalline PLA blocks as compared to those made with amorphous PLA blocks. By changing the size of hydrophobic block the release rate could be significantly modified. Such information can provide the basis on which to engineer the release rate of drugs depending upon the specific application.

What is claimed:

1. An A-B-A triblock copolymer compound comprising A blocks comprising about 17-about 58 weight percent of said compound, each said A block of a formula (II), 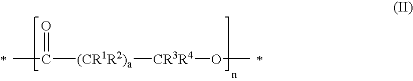
where R¹, R², R³ and R⁴ are moieties independently selected from hydrogen, C₁-about C₆ alkyl, aryl, and X, where X is selected from Cl, Br, F, and I, a is an integer from 0 to about 6 and n is an integer from about 10 to about 300; and a B block comprising about 42-about 83 weight percent poly(ethylene oxide), wherein the elastic modulus at 0.1 Hz of a hydrogel formed from an aqueous medium comprising about 10-about 50 wt % of said compound is about 0.1-about 1,000 kPa.

2. The triblock copolymer of claim 1 wherein said A block is selected from poly(lactic acid), poly(3-hydroxybutyrate), poly(4-hydroxybutyrate), poly(3-hydroxyvalerate), poly(caprolactone), poly(valerolactone) and combinations thereof.

3. The triblock copolymer of claim 2 wherein said A block is selected from poly(L-lactic acid), poly(D,L-lactic acid) and a combination thereof.

4. The triblock copolymer of claim 1 wherein the elastic modulus at 0.1 Hz of a hydrogel formed from an aqueous solution of about 10-about 30 weight percent of said compound is about 0.1-about 10 kPa.

5. The copolymer of claim 1 wherein said A block comprises poly(lactic acid), and said compound is a hydrogel in an aqueous medium, said hydrogel comprising a hydrophobic therapeutic agent.

6. The copolymer of claim 5 wherein said A block of said hydrogel compound is at least partially crystalline.

7. An A-B-A triblock copolymer compound comprising poly(lactic acid) A blocks, each said A block comprising (C(O)CH(CH₃)O)ₘ, wherein n is an integer ranging from about 10 to about 50; and a B block comprising poly(ethylene oxide), wherein the ratio of the degree of polymerization of poly(ethylene oxide) to the degree of polymerization of poly(lactic acid) ranges from about 2.0 to about 8.0.

8. The compound of claim 7 wherein said degree of polymerization of poly(lactic acid) ranges from about 25 to about 75.

9. The compound of claim 7 wherein the molecular weight of poly(ethylene oxide) ranges from about 4,000 to about 16,000.

10. The compound of claim 7 where said A block at least partially comprises poly(L-lactic acid).

11. The compound of claim 7 comprising a hydrogel in an aqueous medium, said hydrogel with an elastic modulus ranging from about 0.1 to about 50 kPa.

12. The compound of claim 7 comprising a hydrogel in an aqueous medium, said hydrogel comprising a therapeutic agent with an affinity for said A block.

13. The compound of claim 12 wherein said A block of said hydrogel is at least partially crystalline.

14. The compound of claim 12 wherein said hydrogel comprises an agent selected from sulindac and tetracaine.

15. A method of a designing and synthesizing a biodegradable A-B-A triblock copolymer having desired properties, comprising the steps of:
selecting a copolymer molecular weight;
selecting a poly(ethylene oxide) B block length;
selecting a ratio of the degree of polymerization of poly(ethylene oxide) to the degree of polymerization of poly(lactic acid);
selecting an A block comprising at least one of poly(L-lactic acid) and poly(D,L-lactic acid) and
synthesizing the triblock copolymer.

16. The method of claim 14 wherein selection of a copolymer molecular weight comprises evaluating PEO weight percent, and selecting overall copolymer elastic modulus.

17. The method of claim 15 wherein selection of a ratio of the degree of polymerization of poly(ethylene oxide) to the degree of polymerization of poly(lactic acid) comprises determining the weight percent of each polymer block for a desired elastic modulus.

18. The method of claim 15 wherein selection of at least one of poly(L-lactic acid) or poly(D,L-lactic acid) comprises selecting a desired drug release rate, and selecting a desired percent of crystallinity.

19. A method of using crystallinity to affect drug release from a poly(lactic acid)-poly(ethylene oxide)-poly(lactic acid) triblock copolymer, said method comprising:
providing a poly(lactic acid)-poly(ethylene oxide)-poly(lactic acid) triblock copolymer compound, said compound comprising a poly(lactic acid) block comprising at least one of L-lactic acid monomers, D-lactic acid monomers and a combination thereof, said poly(lactic acid) block having a crystallinity, said compound in an aqueous medium and in an amount at partially sufficient for gel formation; and
contacting said compound with a therapeutic agent having an affinity for said poly(lactic acid) block.

20. The method of claim 19 wherein said poly(lactic acid) block comprises poly(L-lactic acid).

21. The method of claim 20 wherein an increase in poly(L-lactic acid) content of said compound increases the rate of release of said agent.

22. The method of claim 19 wherein said poly(lactic acid) block comprises a combination of L- and D-lactic acid monomers.

23. The method of claim 22 wherein increasing the D-lactic acid content of said poly(lactic acid) block decreases the rate of release of said agent.

24. The method of claim 19 wherein said agent is selected from sulindac and tetracaine.
